(19) United States
(12) Patent Application Publication
(10) Pub. No.: US 2010/0168176 A1
(43) Pub. Date: Jul. 1, 2010

(54) NOVEL COMPOUNDS, PHARMACEUTICAL COMPOSITIONS CONTAINING SAME, AND METHODS OF USE FOR SAME

(75) Inventors: Craig Townsend, Baltimore, MD (US); Francis Kuhajda, Baltimore, MD (US); Kandasamy Subburaj, Baltimore, MD (US); Jill Marie Sturdivant, Chapel Hill, NC (US)

Correspondence Address: Fox Rothschild LLP, Phlla, Biotech Group, 2000 Market Street, Philadelphia, PA, 19103, US

(73) Assignees: FASGEN LLC, Baltimore, MD (US); THE JOHNS HOPKINS UNIVERSITY, Baltimore, MD (US)

(21) Appl. No.: 12/513,888

(22) PCT Filed: Nov. 8, 2007
(86) PCT No.: PCT/US2007/023552

§ 371 Date: Jan. 4, 2010

Related U.S. Application Data
(60) Provisional application No. 60/857,513, filed on Nov. 8, 2006.

(51) Int. Cl. A61K 31/4436; C07D 333/34; C07D 277/82; C07D 409/12; A61K 31/381; A61K 31/427; A61P 31/04

(52) U.S. Cl. 514/336; 549/65; 548/161; 546/280.4; 514/445; 514/370

(57) ABSTRACT

Compounds having the following general formula, pharmaceutical compositions comprising the compounds, and methods of treating cancer, obesity, and microbial infections using such compositions: wherein: R1=H, C1-C20 alkyl, cycloalkyl, alkenyl, aryl, arylalkyl, or alkylaryl, cyanomethyl, —OCH3, OC(O)CH3 or OC(O)CF3 R2=-OCH2C(O)NHNH—R5, where R5 is (a) phenyl, optionally substituted with one or more of halogen, C1-C8 alkyl, optionally substituted with halogen, —OH, —OR6, where R6 is C1-C8 alkyl, optionally substituted with halogen, or (b) 2-, 3-, or 4-pyridyl, optionally substituted with halogen, —OH, —OR6, where R6 is C1-C8 alkyl, optionally substituted with halogen, or (c) a heterocycle selected from the group consisting of imidazole, thiazole, benzimidazole, benzoxazole, benzthiazole, tetrazole, triazole, and aminothiazole; or (d) —C(O)R7, where R7 is a C1-C20 alkyl, cycloalkyl, alkenyl, aryl, arylalkyl, or alkylaryl, or a heterocycle selected from the group consisting of pyridyl, imidazole, thiazole, benzimidizole, benzoxazole, benzthiazole, tetrazole, triazole, and aminothiazole; and R3 and R4, the same or different from each other, are C1-C20 alkyl, cycloalkyl, alkenyl, aryl, arylalkyl, or alkylaryl.

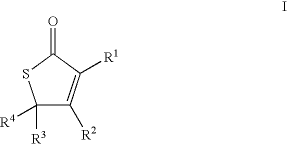

### BACKGROUND OF THE INVENTION

### Fatty Acid Synthase

Fatty acids have three primary roles in the physiology of cells. First, they are the building bocks of biological membranes. Second, fatty acid derivatives serve as hormones and intracellular messengers. Third, and of particular importance to the present invention, fatty acids are fuel molecules that can be stored in adipose tissue as triacylglycerols, which are also known as neutral fats.

There are four primary enzymes involved in the fatty acid synthetic pathway, fatty acid synthase (FAS), acetyl CoA carboxylase (ACC), malic enzyme, and citrate lyase. The principal enzyme, FAS, catalyzes the NADPH-dependent condensation of the precursors malonyl-CoA and acetyl CoA to produce fatty acids. NADPH is a reducing agent that generally serves as the essential electron donor at two points in the reaction cycle of FAS. The other three enzymes (i.e., ACC, malic enzyme, and citrate lyase) produce the necessary precursors. Other enzymes, for example the enzymes that produce NADPH, are also involved in fatty acid synthesis.

FAS has an Enzyme Commission (E.C.) No. 2.3.1.85 and is also known as fatty acid synthase, fatty acid ligase, as well as its systematic name acyl-CoA:malonyl-CoA C-acyltransferase (decarboxylating, oxoacyl- and enoyl-reducing and thioester-hydrolysing). There are seven distinct enzymes—or catalytic domains—involved in the FAS catalyzed synthesis of fatty acids: acetyl transacylase, malonyl transacylase, beta-ketoacyl synthetase (condensing enzyme), beta-ketoacyl reductase, beta-hydroxyacyl dehydrase, enoyl reductase, and thioesterase. (Wakil, S. J., Biochemistry, 28: 4523-4530, 1989). All seven of these enzymes together form FAS.

Although the FAS catalyzed synthesis of fatty acids is similar in lower organisms, such as, for example, and in higher organisms, humans for example, there are some important differences. In bacteria, the seven enzymatic reactions are carried out by seven separate polypeptides that are non-associated. This is classified as Type II FAS. In contrast, the enzymatic reactions in mycobacteria, yeast and humans are carried out by multifunctional polypeptides. For example, yeast have a complex composed of two separate polypeptides whereas in mycobacterium and humans, all seven reactions are carried out by a single polypeptide. These are classified as Type I FAS.

FAS Inhibitors

Various compounds have been shown to inhibit fatty acid synthase (FAS). FAS inhibitors can be identified by the ability of a compound to inhibit the enzymatic activity of purified FAS. FAS activity can be assayed by measuring the incorporation of radiolabeled precursor (i.e., acetyl CoA or malonyl-CoA) into fatty acids or by spectrophotometrically measuring the oxidation of NADPH. (Dils, et al., Methods Enzymol., 35:74-83).

Table 1, set forth below, lists several FAS inhibitors.

TABLE 1
Representative Inhibitors Of The Enzymes Of The Fatty Acid Synthesis Pathway

| Inhibitors of Fatty Acid Synthase | |
|---|---|
| 1,3-dibromopropanone | cerulenin |
| Ellman's reagent (5,5′-dithiobis(2-nitrobenzoic | phenylcerulenin |
| acid), DTNB) | melarsoprol |
| 4-(4′-chlorobenzyloxy) benzyl nicotinate (KCD- | iodoacetate |
| 232) | phenylarsineoxide |
| 4-(4′-chlorobenzyloxy) benzoic acid (MII) | pentostam |
| 2(5(4-chlorophenyl)pentyl)oxirane-2- | melittin |
| carboxylate (POCA) and its CoA derivative | thiolactomycin |
| ethoxyformic anhydride | |
| Inhibitors for citrate lyase | Inhibitors for malic enzyme |
| (−) hydroxycitrate | periodate-oxidized 3-aminopyridine adenine |
| | dinucleotide phosphate |
| S-carboxymethyl-CoA | 5,5′-dithiobis(2-nitrobenzoic acid) |
| radicicol | p-hydroxymercuribenzoate |
| | N-ethylmaleimide |
| | oxalyl thiol esters such as S-oxalylglutathione |
| | gossypol |
| | phenylglyoxal |
| | 2,3-butanedione |
| | bromopyruvate |
| | pregnenolone |
| Inhibitors for alkynyl CoA carboxylase | |
| sethoxydim | 9-decenyl-1-pentenedioic acid |
| haloxyfop and its CoA ester | decanyl-2-pentenedioic acid |
| diclofop and its CoA ester | decanyl-1-pentenedioic acid |
| clethodim | (S)-ibuprofenyl-CoA |
| alloxydim | (R)-ibuprofenyl-CoA |
| trifop | fluazifop and its CoA ester |
| clofibric acid | clofop |
| 2,4-D-mecopropdalapon | 5-(tetradecycloxy)-2-furoic acid |
| 2-alkyl glutarate | beta, beta′-tetramethylhexadecanedioic acid |
| 2-tetradecanylglutarate (TDG) | tralkoxydim |
| 2-octylglutaric acid | free or monothioester of beta, beta prime- |
| N6,02-dibutyryl adenosine cyclic 3′,5′- | methyl-substituted hexadecanedioic acid |
| monophosphate | (MEDICA 16) |
| N2,02-dibutyryl guanosine cyclic 3′,5′- | alpha-cyano-4-hydroxycinnamate |
| monophosphate | S-(4-bromo-2,3-dioxobutyl)-CoA |
| CoA derivative of 5-(tetradecyloxy)-2-furoic | p-hydroxymercuribenzoate (PHMB) |
| acid (TOFA) | N6,02-dibutyryl adenosine cyclic 3′,5′- |
| 2,3,7,8-tetrachlorodibenzo-p-dioxin | monophosphate |

Of the four enzymes in the fatty acid synthetic pathway, FAS is the preferred target for inhibition because it acts only within the pathway to fatty acids, while the other three enzymes are implicated in other cellular functions. Therefore, inhibition of one of the other three enzymes is more likely to affect normal cells. Of the seven enzymatic steps carried out by FAS, the step catalyzed by the condensing enzyme (i.e., beta-ketoacyl synthetase) and the enoyl reductase have been the most common candidates for inhibitors that reduce or stop fatty acid synthesis. The condensing enzyme of the FAS complex is well characterized in terms of structure and function. The active site of the condensing enzyme contains a critical cysteine thiol, which is the target of antilipidemic reagents, such as, for example, the inhibitor cerulenin.

Preferred inhibitors of the condensing enzyme include a wide range of chemical compounds, including alkylating agents, oxidants, and reagents capable of undergoing disulphide exchange. The binding pocket of the enzyme prefers long chain, E, E, dienes.

In principal, a reagent containing the sidechain diene and a group which exhibits reactivity with thiolate anions could be a good inhibitor of the condensing enzyme. Cerulenin [(2S,3R)-2,3-epoxy-4-oxo-7,10 dodecadienoyl amide] is an example:

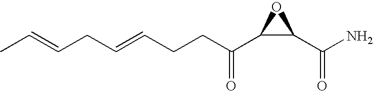

Cerulenin covalently binds to the critical cysteine thiol group in the active site of the condensing enzyme of fatty acid synthase, inactivating this key enzymatic step (Funabashi, et al., J. Biochem., 105:751-755, 1989). While cerulenin hag been noted to possess other activities, these either occur in microorganisms which may not be relevant models of human cells (e.g., inhibition of cholesterol synthesis in fungi, Omura (1976), Bacteriol. Rev., 40:681-697; or diminished RNA synthesis in viruses, Perez, et al. (1991), FEBS, 280: 129-133), occur at a substantially higher drug concentrations (inhibition of viral HIV protease at 5 mg/ml, Moelling, et al. (1990), FEBS, 261:373-377) or may be the direct result of the inhibition of endogenous fatty acid synthesis (inhibition of antigen processing in B lymphocytes and macrophages, Falo, et al. (1987), J. Immunol., 139:3918-3923). Some data suggest that cerulenin does not specifically inhibit myristoylation of proteins (Simon, et al., J. Biol. Chem., 267:3922-3931, 1992).

Several more FAS inhibitors are disclosed in U.S. Pat. No. 5,614,551, the disclosure of which is hereby incorporated by reference. Included are inhibitors of fatty acid synthase, citrate lyase, acetyl CoA carboxylase, and malic enzyme.

Tomoda and colleagues (Tomoda et. al., Biochim. Biophys. Act 921:595-598 1987; Omura el. al., J. Antibiotics 39:1211-1218 1986) describe Triacsin C (sometimes termed WS-1228A), a naturally occurring acyl-CoA synthetase inhibitor, which is a product of Streptomyces sp. SK-1894. The chemical structure of Triacsin C is 1-hydroxy-3-(E, E, E-2′,4′,7′-undecatrienylidine) triazene. Triacsin C causes 50% inhibition of rat liver acyl-CoA synthetase at 8.7 μM; a related compound, Triacsin A, inhibits acyl CoA-synthetase by a mechanism which is competitive with long-chain fatty acids. Inhibition of acyl-CoA synthetase is toxic to animal cells. Tomoda et al. (Tomoda el. al., J. Biol. Chem. 266:4214-4219, 1991) teaches that Triacsin C causes growth inhibition in Raji cells at 1.0 μM, and have also been shown to inhibit growth of Vero and Hela cells. Tomoda el. al. further teaches that acyl-CoA synthetase is essential in animal cells and that inhibition of the enzyme has lethal effects.

A family of compounds (gamma-substituted-alpha-methylene-beta-carboxy-gamma-butyrolactones) has been shown in U.S. Pat. No. 5,981,575 (the disclosure of which is hereby incorporated by reference) to inhibit fatty acid synthesis, inhibit growth of tumor cells, and induce weight loss. The compounds disclosed in the '575 Patent have several advantages over the natural product cerulenin for therapeutic applications: [1] they do not contain the highly reactive epoxide group of cerulenin, [2] they are stable and soluble in aqueous solution, [3] they can be produced by a two-step synthetic reaction and thus easily produced in large quantities, and [4] they are easily tritiated to high specific activity for biochemical and pharmacological analyses. The synthesis of this family of compounds, which are fatty acid synthase inhibitors, is described in the '575 Patent, as is their use as a means to treat tumor cells expressing FAS, and their use as a means to reduce body weight. The '575 Patent also discloses the use of any fatty acid synthase inhibitors to systematically reduce adipocyte mass (adipocyte cell number or size) as a means to reduce body weight.

The primary sites for fatty acid synthesis in mice and humans are the liver (see Roncari, Can. J. Biochem., 52:221-230, 1974; Triscari et al., 1985, Metabolism, 34:580-7; Barakat et al., 1991, Metabolism, 40:280-5), lactating mammary glands (see Thompson, et al., Pediatr. Res., 19:139-143, 1985) and adipose tissue (Goldrick et al., 1974, Clin. Sci. Mol. Med., 46:469-79).

Inhibitors of Fatty Acid Synthesis as Antimicrobial Agents

Cerulenin was originally isolated as a potential antifungal antibiotic from the culture broth of Cephalosporium caerulens. Structurally cerulenin has been characterized as (2R,3S)-epoxy-4-oxo-7,10-trans,trans-dodecanoic acid amide. Its mechanism of action has been shown to be inhibition, through irreversible binding, of beta-ketoacyl-ACP synthase, the condensing enzyme required for the biosynthesis of fatty acids. Cerulenin has been categorized as an antifungal, primarily against Candida and Saccharomyces sp. In addition, some in vitro activity has been shown against some bacteria, actinomycetes, and mycobacteria, although no activity was found against Mycobacterium tuberculosis. The activity of fatty acid synthesis inhibitors and cerulenin in particular has not been evaluated against protozoa such as Toxoplasma gondii or other infectious eucaryotic pathogens such as Pneumocystis carinii, Giardia lamblia, Plasmodium sp., Trichomonas vaginalis, Cryptosporidium, Trypanosoma, Leishmania, and Schistosoma.

Infectious diseases which are particularly susceptible to treatment are diseases which cause lesions in externally accessible surfaces of the infected animal. Externally accessible surfaces include all surfaces that may be reached by non-invasive means (without cutting or puncturing the skin), including the skin surface itself, mucus membranes, such as those covering nasal, oral, gastrointestinal, or urogenital surfaces, and pulmonary surfaces, such as the alveolar sacs. Susceptible diseases include: (1) cutaneous mycoses or tineas, especially if caused by Microsporum, Trichophyton, Epidermophyton, or Mucocutaneous candidiasis; (2) mucotic keratitis, especially if caused by Aspergillus, Fusarium or Candida; (3) amoebic keratitis, especially if caused by Acanthamoeba; (4) gastrointestinal disease, especially if caused by Giardia lamblia, Entamoeba, Cryptosporidium, Microsporidium, or Candida (most commonly in immunocompromised animals); (5) urogenital infection, especially if caused by Candida albicans or Trichomonas vaginalis; and (6) pulmonary disease, especially if caused by Mycobacterium tuberculosis, Aspergillus, or Pneumocystis carinii. Infectious organisms that are susceptible to treatment with fatty acid synthesis inhibitors include Mycobacterium tuberculosis, especially multiply-drug resistant strains, and protozoa such as Toxoplasma.

Any compound that inhibits fatty acid synthesis may be used to inhibit microbial cell growth. However, compounds administered to a patient must not be equally toxic to both patient and the target microbial cells. Accordingly, it is beneficial to select inhibitors that only, or predominantly, affect target microbial cells.

Eukaryotic microbial cells which are dependent on their own endogenously synthesized fatty acid will express Type I FAS. This is shown both by the fact that FAS inhibitors are growth inhibitory and by the fact that exogenously added fatty acids can protect normal patient cells but not these microbial cells from FAS inhibitors. Therefore, agents which prevent synthesis of fatty acids by the cell may be used to treat infections. In eukaryotes, fatty acids are synthesized by Type I FAS using the substrates acetyl CoA, malonyl CoA and NADPH. Thus, other enzymes which can feed substrates into this pathway may also effect the rate of fatty acid synthesis and thus be important in microbes that depend on endogenously synthesized fatty acid. Inhibition of the expression or activity of any of these enzymes will effect growth of the microbial cells that are dependent upon endogenously synthesized fatty acid.

The product of Type I FAS differs in various organisms. For example, in the fungus S. cerevisiae the products are predominately palmitate and stearate esterified to coenzyme-A. In Mycobacterium smegmatis, the products are saturated fatty acid CoA esters ranging in length from 16 to 24 carbons. These lipids are often further processed to fulfill the cells need for various lipid components.

Inhibition of key steps in down-stream processing or utilization of fatty acids may be expected to inhibit cell function, whether the cell depends on endogenous fatty acid or utilizes fatty acid supplied from outside the cell, and so inhibitors of these down-stream steps may not be sufficiently selective for microbial cells that depend on endogenous fatty acid. However, it has been discovered that administration of Type I fatty acid synthesis inhibitor to such microbes makes them more sensitive to inhibition by inhibitors of down-stream fatty acid processing and/or utilization. Because of this synergy, administration of a fatty acid synthesis inhibitor in combination with one or more inhibitors of down-stream steps in lipid biosynthesis and/or utilization will selectively affect microbial cells that depend on endogenously synthesized fatty acid. Preferred combinations include an inhibitor of FAS and acetyl CoA carboxylase, or FAS and an inhibitor of MAS.

When it has been determined that a mammal is infected with cells of an organism which expresses Type I FAS, or if FAS has been found in a biological fluid from a patient, the mammal or patient may be treated by administering a fatty acid synthesis inhibitor (U.S. Pat. No. 5,614,551).

The use of FAS inhibitors to inhibit the growth of cancer cells is described in U.S. Pat. No. 5,759,837, the disclosure of which is hereby incorporated by reference. That application does not describe or disclose any of the compounds disclosed herein.

One class of compounds which can act as FAS inhibitors is disclosed in WO 2004/005277, the disclosure of which is hereby incorporated by reference. That application does not describe or disclose any of the compounds claimed herein.

### SUMMARY OF THE INVENTION

A new class of compounds has been discovered which has a variety of therapeutically valuable properties, eg. FAS-inhibition, and anti-cancer and anti-microbial properties.

It is an object of this invention to provide a method of inducing weight loss in animals and humans by administering a pharmaceutical composition comprising a pharmaceutical diluent and a compound of formula I.

It is a further object of the invention to provide a method of inhibiting fatty acid synthase activity in humans or animals by administering a pharmaceutical composition comprising a pharmaceutical diluent and a compound of formula I.

It is a further object of this invention to provide a method of treating cancer in animals and humans by administering a pharmaceutical composition comprising a pharmaceutical diluent and a compound of formula I.

It is still a further object of this invention to provide a method of preventing the growth of cancer cells in animals and humans by administering a pharmaceutical composition comprising a pharmaceutical diluent and a compound of formula I.

It is a further object of this invention to provide a method of inhibiting growth of invasive microbial cells by administering a pharmaceutical composition comprising a pharmaceutical diluent and a compound of formula I.

### BRIEF DESCRIPTION OF THE DRAWINGS

FIG. 1 show synthetic schemes for making compounds and intermediates pertinent to the invention.

FIG. 2 shows a synthetic scheme for making a compound under the invention.

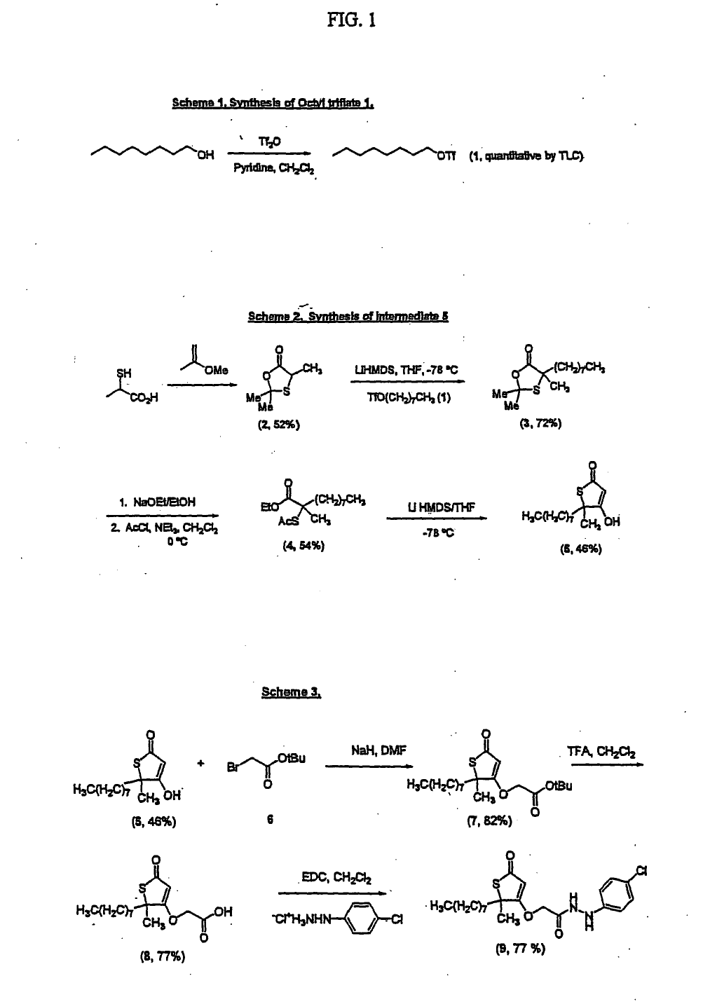

### DETAILED DESCRIPTION OF THE INVENTION

The compounds of the invention can be prepared by conventional means. The synthesis of a number of the compounds is described in the examples. The compounds may be useful for the treatment of obesity, cancer, or microbially-based infections.

One embodiment of the invention is compounds having the following general formula:

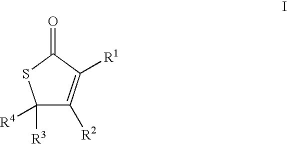

wherein:
R¹═H, C₁-C₂₀ alkyl, cycloalkyl, alkenyl, aryl, arylalkyl, or alkylaryl, cyanomethyl, —OCH₃, —OC(O)CH₃ or —OC(O)CF₃
R²═—OCH₂C(O)NHNH—R⁵, where R⁵ is
   (a) phenyl, optionally substituted with one or more of halogen, C₁-C₈ alkyl, optionally substituted with halogen, —OH, —OR⁶, where R⁶ is C₁-C₈ alkyl, optionally substituted with halogen, or
   (b) 2-, 3-, or 4-pyridyl, optionally substituted with halogen, —OH, —OR⁶, where R⁶ is C₁-C₈ alkyl, optionally substituted with halogen, or
   (c) a heterocycle selected from the group consisting of imidazole, thiazole, benzimidazole, benzoxazole, benzthiazole, tetrazole, triazole, and aminothiazole; or
   (d) —C(O)R⁷, where R⁷ is a C₁-C₂₀ alkyl, cycloalkyl, alkenyl, aryl, arylalkyl, or alkylaryl, or a heterocycle selected from the group consisting of pyridyl, imidazole, thiazole, benzimidizole, benzoxazole, benzthiazole, tetrazole, triazole, and aminothiazole; and
R³ and R⁴, the same or different from each other, are C₁-C₂₀ alkyl, cycloalkyl, alkenyl, aryl, arylalkyl, or alkylaryl;
   with the proviso that when R¹ is —H, —OCH₃, or —OC(O)CF₃ and R³ is —(CH₂)₇CH₃, then R² is not —OCH₂C(O)NHNH—R⁵, where R⁵ is -p-C₆H₄Cl, —C(O)CH₃, or

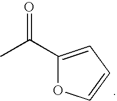

It should be understood that, when applicable, the keto-tautomeric form of the foregoing compounds is also included in formula I.

In a preferred embodiment, R¹ is H.

In another preferred embodiment R⁵ is C₁-C₁₀ alkyl, cycloalkyl, alkenyl, aryl, arylalkyl, or alkylaryl.

In another preferred embodiment, R³ is —H or —CH₃.

In another preferred embodiment, R⁴ is n-C₆-C₈ alkyl.

In another preferred embodiment, R⁶ is C₁-C₁₀ alkyl.

Another embodiment of this invention is a pharmaceutical composition comprising a pharmaceutical diluent and a compound of formula I.

The compositions of the present invention can be presented for administration to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. As used in this specification, the terms “pharmaceutical diluent” and “pharmaceutical carrier,” have the same meaning. For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar or another sweetener, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

The clinical therapeutic indications envisioned for the compounds of the invention include: (1) infections due to invasive micro-organisms such as staphylococci and enterococci; (2) cancers arising in many tissues whose cells over-express fatty acid synthase, and (3) obesity due to the ingestion of excess calories. Dose and duration of therapy will depend on a variety of factors, including (1) the patient's age, body weight, and organ function (e.g., liver and kidney function); (2) the nature and extent of the disease process to be treated, as well as any existing significant co-morbidity and concomitant medications being taken, and (3) drug-related parameters such as the route of administration, the frequency and duration of dosing necessary to effect a cure, and the therapeutic index of the drug. In general, the dose will be chosen to achieve serum levels of 1 ng/ml to 100 ng/ml with the goal of attaining effective concentrations at the target site of approximately 1 μg/ml to 10 μg/ml.

### EXAMPLES

The invention will be illustrated, but not limited, by the following examples:

A series of compounds according to the invention were synthesized as described below. Biological activity of certain compounds was profiled as follows: The compounds were tested for at least some of the following: [1] inhibition of purified human FAS, [2] inhibition of fatty acid synthesis activity in whole cells and [3] cytotoxicity against cultured MCF-7 human breast cancer cells, known to possess high levels of FAS and fatty acid synthesis activity, using the crystal violet and XTT assays. Select compounds with low levels of cytotoxicity were then tested for weight loss in Balb/C mice. Certain compounds were also tested for activity against gram positive and/or negative bacteria.

### Chemical Synthesis of Compounds

### Synthesis of TLM Derivatives Bearing O-Acetic Acid Hydrazides

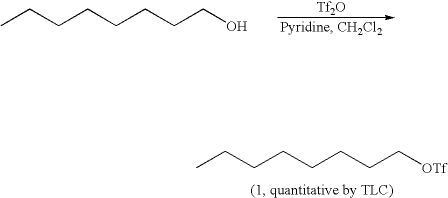

Octyl triflate (1). To octanol (4.6 g, 35.3 mmol) in CH₂Cl₂ (212 mL) cooled to −40° C. was added pyridine (freshly distilled from CaH₂, 3.28 mL, 40.6 mmol), and triflic anhydride (6.41 mL, 38.1 mmol), and the solution was allowed to stir for 20 min at −40° C. Then the reaction mixture was slowly allowed to warm up to room temperature over 3 h. The white solid was then filtered through Celite, which was washed with pentane (2×70 mL). Most of the solvents were evaporated leaving approximately 5-10 mL of solvent and a white precipitate present. Hot pentane (70 mL) was added and this mixture was filtered to remove any remaining pyridine salts. The filtrate was again evaporated to give a clear pale orange oil 1 (quantitative by TLC, rf=0.64 10% EtOAc/Hex) which was used immediately.

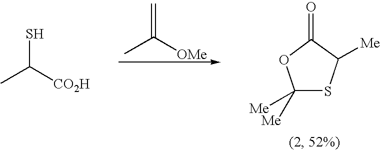

2,2,4-Trimethyl-[1,3]oxathiolan-5-one (2). To thiolactic acid (14.0 g, 132.0 mmol) cooled to 0° C. was added 2-methoxypropene (50.5 mL, 528 mmol) dropwise using an addition funnel. The solution was allowed to warm to room temperature, then heated to reflux for 48 h. After cooling to room temperature, Et₂O (200 mL) was added and this mixture was extracted with Na₂CO₃ (1N, 3×150 mL), and washed with brine (2×100 mL). The combined organics were dried (MgSO₄), filtered and evaporated to give a crude yellow oil, which was distilled (H₂O aspirator pressure, 25-35 torr) at 80-95° C. to give pure 2 (9.9 g, 52%). ¹H NMR (300 MHz, CDCl₃) δ 1.56 (d, J=6.9 Hz, 3H), 1.72 (s, 3H), 1.74 (s, 3H), 4.10 (q, J=6.9 Hz, 1H). ¹³C NMR (75 MHz, CDCl₃) δ 17.9, 30.8, 31.4, 42.5, 86.2, 175.0.

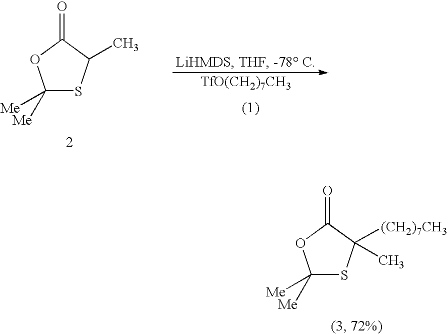

2,2,4-Trimethyl-4-octyl-[1,3]-oxathiolan-5-one (3). To a mixture of LiHMDS (31.7 mL, 31.7 mmol, 1 M in THF) in THF (47 mL) at −78° C. was added 2 (4.3 g, 29.4 mmol) in THF (47 mL) dropwise by cannula, and the resulting yellow solution stirred for 30 min at −78° C. Then, octyl triflate 1 (9.0 g, 35 mmol) in pentane (8 mL) was added slowly at room temperature via cannula to the solution of the enolate at −78° C. After stirring at −78° C. for 2 h, 1 N HCl (200 mL) was added and the solution was extracted with Et₂O (3×75 mL). The combined organics were dried (MgSO₄), filtered and evaporated. Flash chromatography (2% EtOAc/hexanes) gave pure 3 (5.45 g, 72%). ¹H NMR (300 MHz, CDCl₃ δ 0.86 (bs, 3H), 1.25 (m, 10H), 1.63 (s, 3H), 1.73 (s, 3H), 1.80 (s, 3H), 1.5-1.81 (m, 4H); ¹³C NMR (75 MHz, CDCl₃) δ 14.0, 22.6, 25.5, 29.0, 29.1, 29.3, 29.4, 31.8, 32.5, 33.5, 41.4, 58.1, 84.7, 177.7.

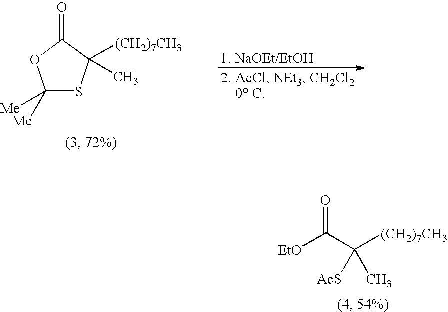

2-Acetylsulfanyl-2-methyl-decanoic acid ethyl ester (4). To 3 (5.33 g, 20.6 mmol) in EtOH (anhydrous, 14.6 mL) was added NaOEt (2.1 M, 12.7 mL, 26.9 mmol) [freshly prepared from Na metal (1.24 g, 54 mmol) in EtOH (24 mL)] and the solution was allowed to stir at room temperature. After 30 min, the solution was poured into NH₄Cl₍ₛₐₜ₎/1 N HCl (100 mL, 3:2) and extracted with Et₂O (3×75 mL). The combined organics were then washed thoroughly with H₂O, dried (MgSO₄), filtered, evaporated and redissolved in CH₂Cl₂ (129 mL). To this precooled solution (0° C.) was added NEt₃ (4.3 mL, 30.9 mmol) and acetyl chloride (3.2 mL, 41.2 mmol). After 40 min at 0° C., NH₄Cl₍ₛₐₜ₎ (200 mL) was added and the solution was extracted with CH₂Cl₂ (3×70 mL). The combined organics were dried (MgSO₄), filtered and evaporated. Flash chromatography (5% EtOAc/hexanes) gave pure 4 (3.1 g, 54%). ¹H NMR (300 MHz, CDCl₃) δ 0.87 (t, J=6.9 Hz, 3H), 1.22-1.27 (m, 15H), 1.61 (s, 3H), 1.75-1.84 (m, 2H), 2.26 (s, 3H), 4.18 (q, J=7.1 Hz, 2H); ¹³C NMR (75 MHz, CDCl₃). δ 13.9, 14.1, 22.6, 23.4, 24.4, 29.1, 29.2, 29.6, 30.3, 31.8, 38.3, 55.8, 61.5, 173.1, 195.8. IR (NaCl) 3430, 1868, 1693, 1644 cm⁻¹; Anal. (C₁₅H₂₈O₃S)C, 62.5; H, 9.78. Found: C, 62.6; H, 9.83.

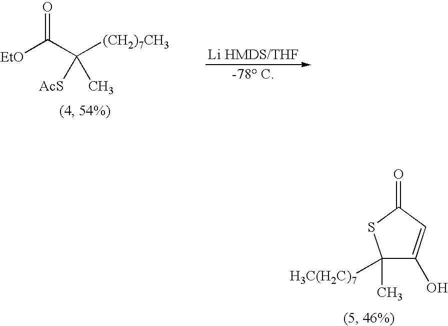

4-Hydroxy-5-methyl-5-octyl-5-H-thiophen-2-one (5). To 4 (3.11 g, 10.8 mmol) in THF (155 mL) at −78° C. was added LiHMDS (13.4 mL, 13.4 mmol, 1.0 M in THF) and the solution was allowed to slowly warm over a 2 h period to −5° C. and then kept at −5° C. for an additional 20 min. The solution was then poured into 1 N HCl (200 mL) and extracted with Et₂O (3×100 mL). The combined organics were dried (MgSO₄), filtered and evaporated. Flash chromatography (20% EtOAc/2% CH₃CO₂H/Hexanes) gave 5 (1.2 g, 46%). ¹H NMR (300 MHz, CDCl₃) (keto-tautomer) δ 0.86 (t, J=6.7 Hz, 3H), 1.19-1.24 (m, 10H), 1.48-1.53 (m, 2H), 1.65 (s, 3H), 137-1.85 (m, 1H), 1.94-2.01 (m, 1H), 3.36 (s, 2H); ¹H NMR (300 MHz, MeOD) (enol tautomer) 0.87-0.89 (m, 3H), 1.29 (m, 10H), 3.29 (s, 3H), 1.81-1.87 (m, 2H); ¹³C NMR (75 MHz, MeOD) (enol tautomer) δ 14.7, 23.8, 26.4, 27.1, 30.5, 30.6, 30.8, 33.2, 39.8, 61.3, 103.1 (m), 189.8, 197.8. IR (NaCl) 3422, 1593 cm⁻¹; Anal. (C₁₃H₂₂O₂S), C, 64.4; H, 9.15. Found: C, 64.3; H, 9.10.

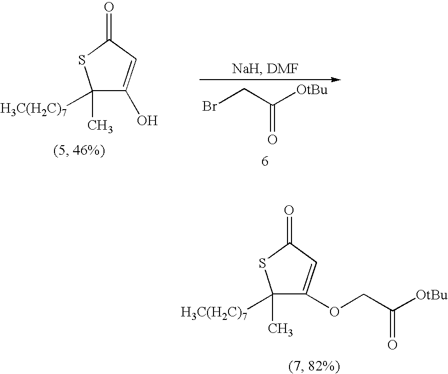

5-Methyl-5-octyl-2-oxo-thiophen-4-yloxy)-acetic acid tert-butyl ester (7). To 5 (1.4 g, 5.8 mmol) in DMF (23 mL) cooled to −40° C. was added NaH (326 mg, 8.15 mmol, 60% in mineral oil) and the solution was allowed to warm and stir at 0° C. for 30 min. t-Butyl bromoacetate 6 (1.29 mL, 8.73 mmol) was then added directly and the mixture was allowed to warm and stir for 3 h at room temperature. NH₄Cl₍ₛₐₜ₎/1 N HCl (6:1, 100 mL) was added and the solution was extracted with Et₂O (3×70 mL). The combined organics were washed with H₂O, dried (MgSO₄), filtered and evaporated. Flash chromatography (15% EtOAc/hexanes) gave pure 7 (1.7 g, 82%). ¹H NMR (300 MHz, CDCl₃) δ 0.86 (t, J=6.9 Hz, 3H), 1.24 (s, 12H), 1.49 (s, 9H), 1.68 (s, 3H), 1.83-1.86 (m, 2H), 4.43 (s, 2H), 5.19 (s, 1H); ¹³C NMR (75 MHz, CDCl₃) 14.0, 22.6, 25.2, 26.3, 28.1, 29.2, 29.3, 29.5, 31.8, 38.9, 59.7, 68.5, 83.4, 102.1, 165.2, 185.5, 193.4. Anal. (C₁₉H₃₂O₄S)C, 64.0; H, 9.05. Found: C, 64.1, H, 9.08.

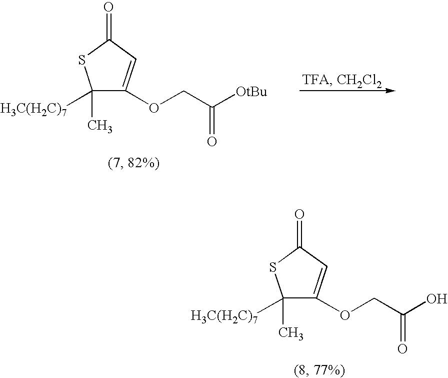

5-Methyl-5-octyl-2-oxo-thiophen-4-yloxy)-acetic acid (8). To 7 (1.7 g, 4.7 mmol) dissolved in CH₂Cl₂ (32 mL) was added trifluoroacetic acid (TFA) (9.1 mL) and the solution was stirred at room temperature for 4-5 h. The solvents were evaporated and the crude material was chromatographed (40% EtOAc/2% CH₃CO₂H/hexanes) to give pure 8 (1.1, 77%). ¹H NMR (300 MHz, CDCl₃) δ 0.86 (t, J=6.9 Hz, 3H), 1.24 (s, 11H), 1.47-1.48 (m, 1H), 1.68 (s, 3H), 1.84-1.88 (m, 2H), 4.62 (s, 2H), 5.31 (s, 1H); ¹³C NMR (75 MHz, CDCl₃) δ 14.1, 22.6, 25.1, 26.1, 29.2, 29.3, 29.5, 31.8, 38.9, 60.1, 67.7, 102.4, 169.8, 185.8, 195.4. IR (NaCl) 3442, 1645 cm⁻¹; Anal. (C₁₅H₂₄O₄S)C, 59.9; H, 8.05. Found: C, 60.0; H, 8.09.

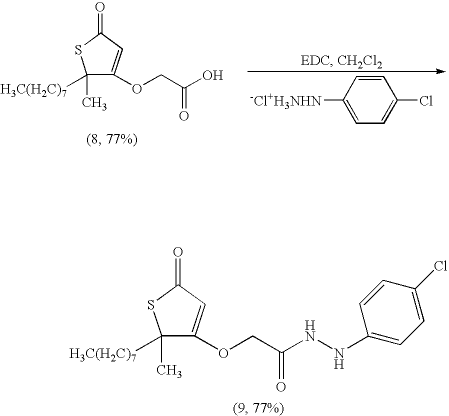

(5-Methyl-5-octyl-2-oxo-thiophen-4-yloxy)-acetic-acid-N′-(4-chlorophenyl)-hydrazide (9). To a cooled solution (0° C.) of 8 (1.1 g, 3.67 mmol) in CH₂Cl₂ (17.3 mL) was added 1-[3-(dimethylamino)propyl]-3-ethylcarbodiimide hydrochloride (EDC) (1.4 g, 7.3 mmol), 4-chlorophenylhydrazine hydrochloride (854 mg, 4.77 mmol), NEt₃ (0.51 mL, 3.67 mmol), and DMAP (67 mg, 0.55 mmol). This mixture was stirred at 0° C. for 30 min, then warmed to room temperature and stirred for 12 h. The solution was poured into NH₄Clₛₐₜ:HCl (1N) (4:1, 100 ml) and extracted with CH₂Cl₂ (3×30 ml). The combined organics were dried (MgSO₄), filtered and evaporated to give crude 9. Flash chromatography [30% EtOAc/Hex (removes byproducts)—then 35% EtOAc/Hex (500 mL)-40% EtOAc/Hex (300 mL)] gave pure 9 (1.2 g, 77%). ¹H NMR (300 MHz, CDCl₃) δ 0.86 (t, J=6 Hz, 3H), 1.24 (m, 11H), 1.46-1.54 (m, 1H), 1.71 (s, 3H), 1.82-1.90 (m, 2H), 4.57 (s, 2H), 5.39 (s, 1H), 6.75 (d, J=8.8 Hz, 2H), 7.18 (d, J=8.8 Hz, 2H), 7.38 (s, 1H), 8.09 (s, 1H); ¹³C NMR (100 MHz, CDCl₃) δ 14.1, 22.6, 25.3, 26.1, 29.2, 29.3, 29.5, 31.8, 38.8, 59.7, 69.7, 103.2, 114.7, 126.4, 145.8, 129.2, 165.9, 184.3, 193.5. IR (NaCl) 2957, 1695, 1658, 1609 cm⁻¹.

### General Procedure A:

To a cooled solution (0° C.) of 8 (0.05 mmol, 1.0 equiv.) in CH₂Cl₂ (1.0 mL) was added 1-[3-(dimethylamino)propyl]-3-ethylcarbodiimide hydrochloride (EDC) (0.1 mmol, 2.0 equiv.), hydrazine derivative (0.065 mmol, 1.3 equiv.), and DMAP (0.0075 mmol, 0.15 equiv.) and triethyl amine (1.0 equiv.) if hydrochloride salt was used in the reaction. The mixture was stirred at 0° C. for 30 min, then warmed to room temperature and stirred for 12 h. The reaction mixture was transferred to a silica gel column packed with CH₂Cl₂. Flash chromatography (20% Ether/CH₂Cl₂) gave pure product.

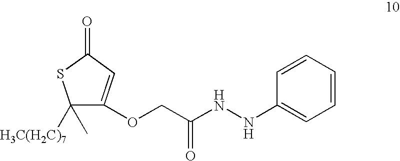

(5-Methyl-5-octyl-2-oxo-thiophen-4-yloxy)-acetic-acid-N′-phenylhydrazide (10). To 8 (15.0 mg, 0.05 mmol) and phenylhydrazine (6.4 μL, 0.065 mmol), following general procedure A compound 10 was obtained (15.0 mg, 77%) as an oil (cis:trans ratio—27:73). ¹H NMR (400 MHz, CDCl₃) δ 0.80 (t, J=8.0 Hz, 3H), 1.10-1.24 (m, 11H), 1.41-1.51 (m, 1H), 1.66 (s, 3H), 1.78-1.84 (m, 2H), 4.50 (s, 2H), 5.33 (s, 1H), 6.75 (dd, J=1.2, 8.0 Hz, 2H), 6.90 (dd, J=8.0, 16.0 Hz, 1H), 7.20 (dd, J=8.0, 16.0 Hz, 2H), 8.03 (s, 1H); Representative peaks for cis compound: 1.62 (s, 3H), 4.75 (s, 2H), 5.13 (s, 1H); ¹³C NMR (100 MHz, CDCl₃) δ 14.1, 22.6, 25.4, 26.3, 29.2, 29.3, 29.5, 31.8, 39.0, 59.4, 70.0, 103.5, 113.6, 122.0, 129.4, 147.0, 165.6, 183.9, 193.0.

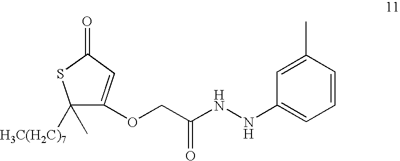

(5-Methyl-5-octyl-2-oxo-thiophen-4-yloxy)-acetic-acid-N′-(3-methylphenyl)-hydrazide (11). To 8 (15.0 mg, 0.05 mmol) and 1-(3-methylphenyl)hydrazine hydrochloride (10.2 mg, 0.065 mmol), following general procedure A compound 11 was obtained (7.0 mg, 35%) as an oil (cis:trans ratio—29:71). ¹H NMR (400 MHz, CDCl₃) δ 0.87 (t, J=8.0 Hz, 3H), 1.19-1.29 (m, 11H), 1.51-1.57 (m, 1H), 1.74 (s, 3H), 1.85-1.91 (m, 2H), 2.30 (s, 3H), 4.59 (s, 2H), 5.14 (s, 1H), 6.62-6.65 (m, 2H), 6.77 (d, J=8.0 Hz, 1H), 7.07-7.19 (m, 1H), 7.93 (s, 1H); Representative peaks for cis compound: 1.69 (s, 3H), 2.33 (s, 3H), 4.82 (s, 2H), 5.23 (s, 1H); ¹³C NMR (100 MHz, CDCl₃) δ 14.1, 21.5, 22.6, 25.0, 26.4, 29.2, 29.4, 29.6, 31.8, 38.5, 59.0, 70.0, 103.0, 110.5, 114.0, 122.5, 129.0, 139.0, 147.0, 165.0, 184.0, 193.0.

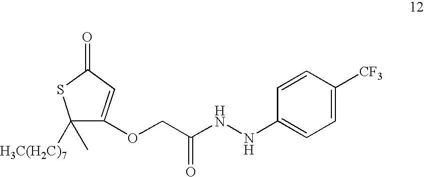

(5-Methyl-5-octyl-2-oxo-thiophen-4-yloxy)-acetic-acid-N′-(4-trifluoromethylphenyl)-hydrazide (12). To 8 (15.0 mg, 0.05 mmol) and 1-[4-(trifluoromethyl)-phenyl]hydrazine hydrochloride (14.0 mg, 0.065 mmol), following general procedure A compound 12 was obtained (12.0 mg, 53%) as an oil (cis:trans ratio—17:83). ¹H NMR (400 MHz, CDCl₃) δ 0.80 (t, J=8.0 Hz, 3H), 1.10-1.25 (m, 11H), 1.45-1.51 (m, 1H), 1.68 (s, 3H), 1.78-1.85 (m, 2H), 4.56 (s, 2H), 5.36 (s, 1H), 6.29 (s, 1H), 6.81 (d, J=8.0 Hz, 2H), 7.43 (d, J=8.0 Hz, 2H), 7.94 (s, 1H); Representative peaks for cis compound: 1.62 (s, 3H), 4.74 (s, 2H), 5.18 (s, 1H).

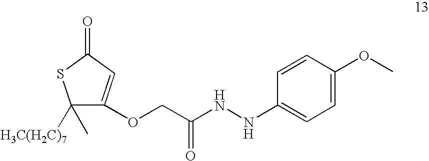

(5-Methyl-5-octyl-2-oxo-thiophen-4-yloxy)-acetic-acid-N′-(4-methoxyphenyl)-hydrazide (13). To 8 (15.0 mg, 0.05 mmol) and 1-(4-methoxyphenyl)hydrazine hydrochloride (11.3 mg, 0.065 mmol), following general procedure A compound 13 was obtained (7.0 mg, 33%) as an oil (cis:trans ratio—25:75). ¹H NMR (400 MHz, CDCl₃) δ 0.80 (t, J=8.0 Hz, 3H), 1.08-1.24 (m, 11H), 1.41-1.51 (m, 1H), 1.66 (s, 3H), 1.80-1.84 (m, 2H), 3.69 (s, 3H), 4.50 (s, 2H), 5.33 (s, 1H), 6.76 (s, 4H), 7.90 (s, 1H); Representative peaks for cis compound: 1.63 (s, 3H), 3.71 (s, 3H), 4.76 (s, 2H), 5.15 (s, 1H); ¹³C NMR (75 MHz, CDCl₃) δ 14.1, 22.6, 25.4, 26.4, 29.2, 29.4, 29.5, 31.8, 39.0, 55.6, 59.4, 69.9, 103.5, 114.3, 114.7, 139.7, 155.3, 165.5, 183.8, 192.9.

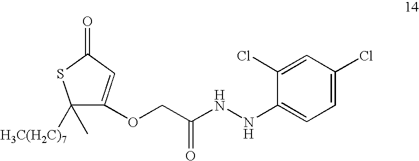

(5-Methyl-5-octyl-2-oxo-thiophen-4-yloxy)-acetic-acid-N′-(2,4-dichlorophenyl)-hydrazide (14). To 8 (19.0 mg, 0.063 mmol) and 1-(2,4-dichlorophenyl)hydrazine hydrochloride (17.5 mg, 0.082 mmol), following general procedure A compound 14 was obtained (17.0 mg, 59%) as a solid (cis:trans ratio—20:80). ¹H NMR (400 MHz, CDCl₃) δ 0.86 (t, J=7.2 Hz, 3H), 1.15-1.31 (m, 11H), 1.42-1.51 (m, 1H), 1.72 (s, 3H), 1.82-1.90 (m, 2H), 4.77 (s, 2H), 5.41 (s, 1H), 6.38 (s, 1H), 6.76 (d, J=8.4 Hz, 1H), 7.14 (dd, J=2.0, 8.4 Hz, 1H), 7.32 (d, J=2.0 Hz, 1H), 8.11 (s, 1H); Representative peaks for cis compound: 1.65 (s, 3H), 4.75 (s, 2H), 5.20 (s, 1H); ¹³C NMR (100 MHz, CDCl₃) δ 14.1, 22.6, 25.4, 26.3, 29.2, 29.4, 29.5, 31.8, 39.0, 59.5, 69.8, 103.5, 114.4, 120.5, 126.5, 127.8, 129.4, 141.9, 165.5, 183.9, 193.0.

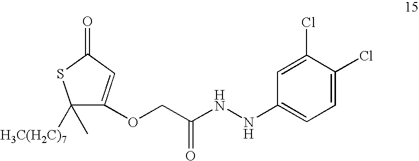

(5-Methyl-5-octyl-2-oxo-thiophen-4-yloxy)-acetic-acid-N′-(3,4-dichlorophenyl)-hydrazide (15). To 8 (19.0 mg, 0.063 mmol) and 1-(3,4-dichlorophenyl)hydrazine hydrochloride (17.5 mg, 0.082 mmol), following general procedure A compound 15 was obtained (12.0 mg, 42%) as a semisolid (cis:trans ratio—17:83). ¹H NMR (400 MHz, CDCl₃) δ 0.80 (t, J=6.8 Hz, 3H), 1.09-1.23 (m, 11H), 1.36-1.53 (m, 1H), 1.67 (s, 3H), 1.77-1.84 (m, 2 H), 4.54 (s, 2H), 5.35 (s, 1H), 6.21 (s, 1H), 6.60 (dd, J=2.4, 8.8 Hz, 1H), 6.84 (d, J=2.4 Hz, 1H), 7.21 (d, J=8.8 Hz, 1H), 8.0 (s, 1H); Representative peaks for cis compound: 1.65 (s, 3H), 4.73 (s, 2H), 5.18 (s, 1H); ¹³C NMR (75 MHz, CDCl₃) δ 14.1, 22.6, 25.4, 26.3, 29.2, 29.4, 29.5, 31.8, 39.0, 59.5, 69.8, 103.5, 113.2, 115.2, 124.8, 130.9, 133.2, 146.7, 165.9, 184.0, 193.2.

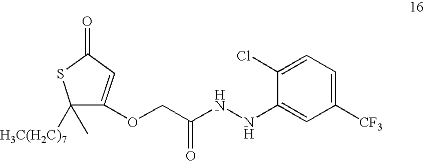

(5-Methyl-5-octyl-2-oxo-thiophen-4-yloxy)-acetic-acid-N′-[2-chloro-5-(trifluoromethyl)phenyl]-hydrazide (16). To 8 (15.0 mg, 0.05 mmol) and 1-[2-chloro-5-(trifluoromethyl)phenyl]hydrazine (13.6 mg, 0.065 mmol), following general procedure A compound 16 was obtained (10.4 mg, 42%) as an oil (cis:trans ratio—14:86). ¹H NMR (300 MHz, CDCl₃) δ 0.80 (t, J=6.6 Hz, 3H), 1.06-1.24 (m, 11H), 1.41-1.50 (m, 1H), 1.67 (s, 3H), 1.76-1.89 (m, 2H), 4.58 (s, 2H), 5.37 (s, 1H), 6.53 (d, J=3.3 Hz, 1H), 6.98 (d, J=1.5 Hz, 1H), 7.07 (dd, J=1.8, 8.4 Hz, 1H), 7.37 (d, J=8.1 Hz, 1H), 8.03 (s, 1H); Representative peaks for cis compound: 1.60 (s, 3H), 4.77 (s, 2H), 5.28 (s, 1H).

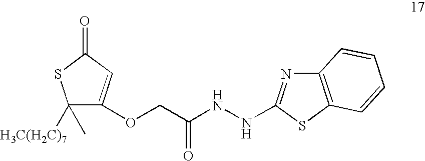

(5-Methyl-5-octyl-2-oxo-thiophen-4-yloxy)-acetic-acid-N′-(2-benzothiazole)-hydrazide (17). To 8 (22.0 mg, 0.07 mmol) and 2-hydrazinobenzothiazole (14.6 mg, 0.09 mmol), following general procedure A compound 17 was obtained (14.0 mg, 45%) as a solid (single isomer). ¹H NMR (400 MHz, CDCl₃) δ 0.88 (t, J=6.4 Hz, 3H), 1.26-1.83 (m, 11H), 1.52 (m, 1H), 1.75 (s, 3H), 1.86-1.99 (m, 2H), 4.86 (s, 2H), 5.22 (s, 2H), 5.32 (s, 1H), 7.36 (t, J=7.2 Hz, 1H), 7.48 (t, J=7.2 Hz, 1H), 7.81 (d, J=8.0 Hz, 1H), 7.85 (d, J=8.0 Hz, 1H); m.p. 151-152° C.

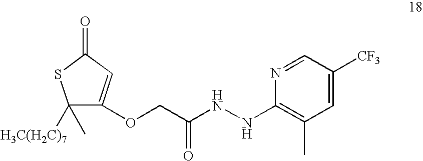

(5-Methyl-5-octyl-2-oxo-thiophen-4-yloxy)-acetic-acid-N′-[6-methyl-4-(trifluoromethyl)-2-pyridyl]-hydrazide (18). To 8 (15.0 mg, 0.05 mmol) and 1-[6-methyl-4-(trifluoromethyl)-2-pyridyl]hydrazine (12.5 mg, 0.065 mmol), following general procedure A compound 18 was obtained (12.5 mg, 53%) as an oil (cis:trans ratio—10:90). ¹H NMR (300 MHz, CDCl₃) δ 0.79 (t, J=8.4 Hz, 3H), 1.10-1.29 (m, 11H), 1.44-1.52 (m, 1H), 1.70 (s, 3H), 1.82-1.89 (m, 2H), 2.41 (s, 3H), 4.57 (s, 2H), 5.37 (s, 1H), 6.59 (s, 1H), 6.81 (s, 1H), 7.31 (bs, 1H), 8.70 (bs, 1H); Representative peaks for cis compound: 1.62 (s, 3H), 4.74 (s, 2H), 5.29 (s, 1H).

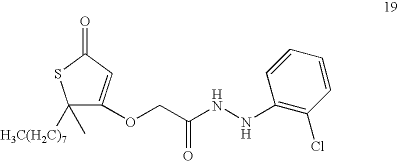

(5-Methyl-5-octyl-2-oxo-thiophen-4-yloxy)-acetic-acid-N′-(2-chlorophenyl)-hydrazide (19). To 8 (98.0 mg, 0.33 mmol) and 2-chlorophenylhythazine hydrochloride (77.0 mg, 0.43 mmol), following general procedure A compound 19 was obtained (91.0 mg, 60%). ¹H NMR (300 MHz, CDCl₃) δ 0.85 (t, J=7.0 Hz, 3H), 1.23 (m, 11H), 1.48-1.51 (m, 1H), 1.69 (s, 3H), 1.81-1.89 (m, 2H), 4.56 (s, 2H), 5.37 (s, 1H), 6.49-6.51 (m, 1H), 6.84-6.94 (m, 2H), 7.12-7.35 (m, 2H), 8.31 (d, J=3.1 Hz, 1H). ¹³C NMR (100 MHz, CDCl₃) δ 14.0, 22.5, 25.3, 26.2, 29.2, 29.3, 29.5, 31.7, 38.9, 59.5, 69.7, 103.3, 113.5, 119.8, 122.0, 122.7, 129.6, 142.9, 165.5, 184.1, 193.3.

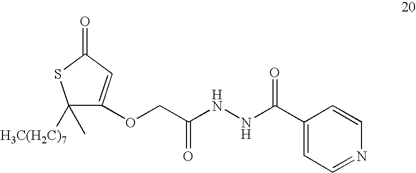

(5-Methyl-5-octyl-2-oxo-thiophen-4-yloxy)-acetic-acid-N′-(4-pyridyl)-hydrazide (20). To 8 (100.0 mg, 0.33 mmol) and isonicotinic hydrazine (59.0 mg, 0.42 mmol), following general procedure A compound 20 was obtained (118.0 mg, 86%) after flash chromatography (5% MeOH/CHCl₃). ¹H NMR. (300 MHz, CDCl₃) δ 0.85 (t, J=7.0 Hz, 3H), 1.23 (m, 11H), 1.44-1.45 (m, 1H), 1.68 (s, 3H), 1.82-1.88 (m, 2H), 4.69 (s, 2H), 5.42 (s, 1H), 7.64 (d, J=5.3 Hz, 2H), 8.67 (m, 2H). ¹³C NMR (100 MHz, CDCl₃) δ 14.0, 22.5, 25.3, 26.2, 29.2, 29.3, 29.5, 31.7, 38.9, 59.5, 69.7, 103.3, 113.5, 119.8, 122.0, 122.7, 129.6, 142.9, 165.5, 184.1, 193.3.

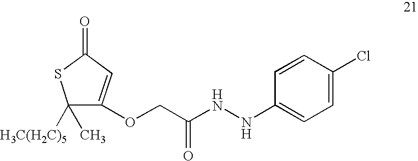

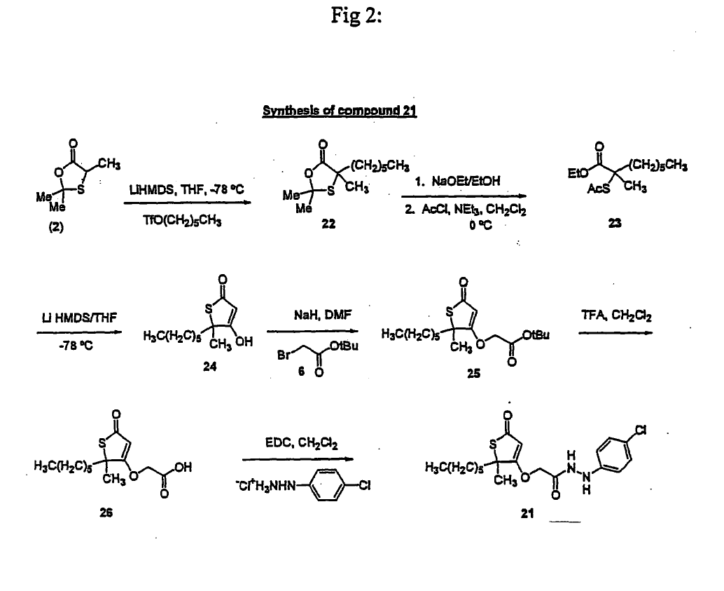

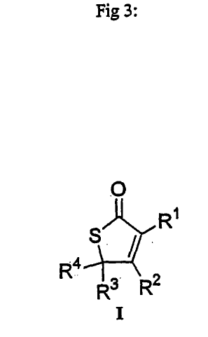

(5-Methyl-5-hexyl-2-oxo-thiophen-4-yloxy)-acetic-acid-M-(4-chlorophenyl)-hydrazide (21). This compound was prepared according to the scheme shown in FIG. 2. To 26 (83.0 mg, 0.29 mmol) and 4-chlorophenylhydrazine hydrochloride (68.0 mg, 0.38 mmol), following general procedure A compound 21 was obtained (34.0 mg, 30%). ¹H NMR (300 MHz, CDCl₃) δ 0.86 (m, 3H), 1.26 (m, 7H), 1.45-1.50 (m, 1H), 1.71 (s, 3H), 1.85-1.90 (m, 2H), 4.57 (s, 2H), 5.39 (s, 1H), 6.74 (d, J=8.8 Hz, 2H), 7.20 (d, J=8.8 Hz, 2H), 7.59 (s, 1H), 8.21 (s, 1H).

### Biological and Biochemical Methods

Purification of FAS from ZR-75-1 Human Breast Cancer Cells.

Human FAS was purified from cultured ZR-75-1 human breast cancer cells obtained from the American Type Culture Collection. The procedure, adapted from Linn et al., 1981, and Kuhajda et al., 1994, utilizes hypotonic lysis, successive polyethyleneglycol (PEG) precipitations, and anion exchange chromatography. ZR-75-1 cells are cultured at 37° C. with 5% CO₂ in RPMI culture medium with 10% fetal bovine serum, penicillin and streptomycin.

Ten T150 flasks of confluent cells are lysed with 1.5 ml lysis buffer (20 mM Tris-HCl, pH 7.5, 1 mM EDTA, 0.1 mM phenylmethanesulfonyl fluoride (PMSF), 0.1% Igepal CA-630) and dounce homogenized on ice for 20 strokes. The lysate is centrifuged in JA-20 rotor (Beckman) at 20,000 rpm for 30 minutes at 4° C. and the supernatant is brought to 42 ml with lysis buffer. A solution of 50% PEG 8000 in lysis buffer is added slowly to the supernatant to a final concentration of 7.5%. After rocking for 60 minutes at 4° C., the solution is centrifuged in JA-20 rotor (Beckman) at 15,000 rpm for 30 minutes at 4° C. Solid PEG 8000 is then added to the supernatant to a final concentration of 15%. After the rocking and centrifugation is repeated as above, the pellet is resuspended overnight at 4° C. in 10 ml of Buffer A (20 mM K₂HPO₄, pH 7.4). After 0.45 μM filtration, the protein solution is applied to a Mono Q 5/5 anion exchange column (Pharmacia). The column is washed for 15 minutes with buffer A at 1 ml/minute, and bound material is eluted with a linear 60-ml gradient over 60 minutes to 1 M KCl. FAS (MW˜270 kD) typically elutes at 0.25 M KCl in three 0.5 ml fractions identified using 4-15% SDS-PAGE with Coomassie G250 stain (Bio-Rad). FAS protein concentration is determined using the Coomassie Plus Protein Assay Reagent (Pierce) according to manufacturer's specifications using BSA as a standard. This procedure results in substantially pure preparations of FAS (>95%) as judged by Coomassie-stained gels.

### Measurement of FAS Enzymatic Activity and Determination of the IC₅₀ of the Compounds

FAS activity is measured by monitoring the malonyl-CoA dependent oxidation of NADPH spectrophotometrically at OD₃₄₀ in 96-well plates (Dils et al and Arslanian et al, 1975). Each well contains 2 μg purified FAS, 100 mM K₂HPO₄, pH 6.5, 1 mM dithiothreitol (Sigma), and 187.5 μM β-NADPH (Sigma). Stock solutions of inhibitors are prepared in DMSO at 2, 1, and 0.5 mg/ml resulting in final concentrations of 20, 10, and 5 μg/ml when 1 μl of stock is added per well. For each experiment, cerulenin (Sigma) is run as a positive control along with DMSO controls, inhibitors, and blanks (no FAS enzyme) all in duplicate.

The assay is performed on a Molecular Devices SpectraMax Plus Spectrophotometer. The plate containing FAS, buffers, inhibitors, and controls are placed in the spectrophotometer heated to 37° C. Using the kinetic protocol, the wells are blanked on duplicate wells containing 100 μl of 100 mM K₂HPO₄, pH 6.5 and the plate is read at OD₃₄₀ at 10 sec intervals for 5 minutes to measure any malonyl-CoA independent oxidation of NADPH. The plate is removed from the spectrophotometer and malonyl-CoA (67.4 μM, final concentration per well) and alkynyl-CoA (61.8 μM, final concentration per well) are added to each well except to the blanks. The plate is read again as above with the kinetic protocol to measure the malonyl-CoA dependent NADPH oxidation. The difference between the Δ OD₃₄₀ for the malonyl-CoA dependent and non-malonyl-CoA dependent NADPH oxidation is the specific FAS activity. Because of the purity of the FAS preparation, non-malonyl-CoA dependent NADPH oxidation is negligible.

The IC₅₀ for the compounds against FAS is determined by plotting the Δ OD₃₄₀ for each inhibitor concentration tested, performing linear regression and computing the best-fit line, r² values, and 95% confidence intervals. The concentration of compound yielding 50% inhibition of FAS is the IC₅₀. Graphs of Δ OD₃₄₀ versus time are plotted by the SOFTmax PRO software (Molecular Devices) for each compound concentration. Computation of linear regression, best-fit line, r², and 95% confidence intervals are calculated using Prism Version 3.0 (Graph Pad Software).

### Crystal Violet Cell Growth Assay

The crystal violet assay measure cell growth but not cytotoxicity. This assay employs crystal violet staining of fixed cells in 96-well plates with subsequent solubilization and measurement of OD₄₉₀ on a spectrophotometer. The OD₄₉₀ corresponds to cell growth per unit time measured. Cells are treated with the compounds of interest or vehicle controls and IC₅₀ for each compound is computed.

To measure the cytotoxicity of specific compounds against cancer cells, 5×10⁴ MCF-7 human breast cancer cells, obtained from the American Type Culture Collection are plated per well in 24 well plates in DMEM medium with 10% fetal bovine serum, penicillin, and streptomycin. Following overnight culture at 37° C. and 5% CO₂, the compounds to be tested, dissolved in DMSO, are added to the wells in 1 μl volume at the following concentrations: 50, 40, 30, 20, and 10 μg/ml in triplicate. Additional concentrations are tested if required. 1 μl of DMSO is added to triplicate wells are the vehicle control. C75 is run at 10, and 5 μg/ml in triplicate as positive controls.

After 72 hours of incubation, cells are stained with 0.5 ml of Crystal Violet stain (0.5% in 25% methanol) in each well. After 10 minutes, wells are rinsed, air dried, and then solubilized with 0.5 ml 10% sodium dodecylsulfate with shaking for 2 hours. Following transfer of 100 μl from each well to a 96-well plate, plates are read at OD₄₉₀ on a Molecular Devices SpectraMax Plus Spectrophotometer Average OD₄₉₀ values are computed using SOFTmax Pro Software (Molecular Devices) and IC₅₀ values are determined by linear regression analysis using Prism version 3.02 (Graph Pad Software, San Diego).

### XTT Cytotoxicity Assay

The XTT assay is a non-radioactive alternative for the [⁵¹Cr] release cytotoxicity assay. XTT is a tetrazolium salt that is reduced to a formazan dye only by metabolically active, viable cells. The reduction of XTT is measured spectrophotometrically as OD₄₉₀-OD₆₅₀.

To measure the cytotoxicity of specific compounds against cancer cells, 9×10³ MCF-7 human breast cancer cells, obtained from the American Type Culture Collection are plated per well in 96 well plates in DMEM medium with 10% fetal bovine serum, insulin, penicillin, and streptomycin. Following overnight culture at 37° C. and 5% CO₂, the compounds to be tested, dissolved in DMSO, are added to the wells in 1 μl volume at the following concentrations: 80, 40, 20, 10, 5, 2.5, 1.25, and 0.625 μg/ml in triplicate. Additional concentrations are tested if required. 1 μl of DMSO is added to triplicate wells are the vehicle control. C75 is run at 40, 20, 10, 15, 12.5, 10, and 5 in triplicate as positive controls.

After 72 hours of incubation, cells are incubated for 4 hours with the XTT reagent as per manufacturer's instructions (Cell Proliferation Kit II (XTT) Roche). Plates are read at OD₄₉₀ and OD₆₅₀ on a Molecular Devices SpectraMax Plus Spectrophotometer. Three wells containing the XTT reagent without cells serve as the plate blank. XTT data are reported as OD₄₉₀-OD₆₅₀. Averages and standard error of the mean are computed using SOFTmax Pro software (Molecular Dynamics).

The IC₅₀ for the compounds is defined as the concentration of drug leading to a 50% reduction in OD₄₀₀-OD₆₅₀ compared to controls. The OD₄₀₀-OD₆₅₀ are computed by the SOFTmax PRO software (Molecular Devices) for each compound concentration. IC₅₀ is calculated by linear regression, plotting the FAS activity as percent of control versus drug concentrations. Linear regression, best-fit line, r², and 95% confidence intervals are determined using Prism Version 3.0 (Graph Pad Software).

Measurement of [¹⁴ C]acetate Incorporation into Total Lipids and Determination of IC₅₀ of Compounds

This assay measures the incorporation of [¹⁴C]acetate into total lipids and is a measure of fatty acid synthesis pathway activity in vitro. It is utilized to measure inhibition of fatty acid synthesis in vitro.

MCF-7 human breast cancer cells cultured as above, are plated at 5×10⁴ cells per well in 24-well plates. Following overnight incubation, the compounds to be tested, solubilized in DMSO, are added at 5, 10, and 20 μg/ml in triplicate, with lower concentrations tested if necessary. DMSO is added to triplicate wells for a vehicle control. C75 is run at 5 and 10 μg/ml in triplicate as positive controls. After 4 hours of incubation, 0.25 μCi of [¹⁴C]acetate (10 μl volume) is added to each well.

After 2 hours of additional incubation, medium is aspirated from the wells and 800 μl of chloroform:methanol (2:1) and 700 μl of 4 mM MgCl₂ is added to each well. Contents of each well are transferred to 1.5 Eppendorf tubes, and spun at full-speed for 2 minutes in a high-speed Eppendorf Microcentrifuge 5415D. After removal of the aqueous (upper) layer, an additional 700 μl of chloroform:methanol (2:1) and 500 μl of 4 mM MgCl₂ are added to each tube and then centrifuged for 1 minutes as above. The aqueous layer is removed with a Pasteur pipette and discarded. An additional 400 μl of chloroform:methanol (2:1) and 200 μl of 4 mM MgCl₂ are added to each tube, then centrifuged and aqueous layer is discarded. The lower (organic) phase is transferred into a scintillation vial and dried at 40° C. under N₂ gas. Once dried, 3 ml of scintillant (APB #NBC5104) is added and vials are counted for ¹⁴C. The Beckman Scintillation counter calculates the average cpm values for triplicates.

The IC₅₀ for the compounds is defined as the concentration of drug leading to a 50% reduction in [¹⁴C]acetate incorporation into lipids compared to controls. This is determined by plotting the average cpm for each inhibitor concentration tested, performing linear regression and computing the best-fit line, r² values, and 95% confidence intervals. The average cpm values are computed by the Beckman scintillation counter (Model LS6500) for each compound concentration. Computation of linear regression, best-fit line, r², and 95% confidence intervals are calculated using Prism Version 3.0 (Graph Pad Software).

### Weight Loss Screen for Novel FAS Inhibitors

Balb/C mice (Jackson Labs) are utilized for the initial weight loss screening. Animals are housed in temperature and 12 hour day/night cycle rooms and fed mouse chow and water ad lib. Three mice are utilized for each compound tested with vehicle controls in triplicate per experiment. For the experiments, mice are housed separately for each compound tested three mice to a cage. Compounds are diluted in DMSO at 10 mg/ml and mice are injected intraperitoneally with 60 mg/kg in approximately 100 μl of DMSO or with vehicle alone. Mice are observed and weighed daily; average weights and standard errors are computed with Excel (Microsoft). The experiment continues until treated animals reach their pretreatment weights.

### Antimicrobial Properties

A broth microdilution assay is used to assess the antimicrobial activity of the compounds. Compounds are tested at twofold serial dilutions, and the concentration that inhibits visible growth (OD₆₀₀ at 10% of control) is defined as the MIC. Microorganisms tested include Staphylococcus aureus (ATCC # 29213), Enterococcus faecalis (ATCC # 29212), Pseudomonas aeruginosa (ATCC # 27853), and Escherichia coli (ATCC # 25922). The assay is performed in two growth media, Mueller Hinton Broth and Trypticase Soy Broth.

A blood (Tsoy/5% sheep blood) agar plate is inoculated from frozen stocks maintained in T soy broth containing 10% glycerol and incubated overnight at 37° C. Colonies are suspended in sterile broth so that the turbidity matches the turbidity of a 0.5 McFarland standard. The inoculum is diluted 1:10 in sterile broth (Mueller Hinton or Trypticase soy) and 195 ul is dispensed per well of a 96-well plate. The compounds to be tested, dissolved in DMSO, are added to the wells in 5 ul volume at the following concentrations: 25, 12.5, 6.25, 3.125, 1.56 and 0.78 ug/ml in duplicate. Additional concentrations are tested if required. 5 ul of DMSO added to duplicate wells are the vehicle control. Serial dilutions of positive control compounds, vancomycin (E. faecalis and S. aureus) and tobramycin (E. coli and P. aeruginosa), are included in each run.

After 24 hours of incubation at 37° C., plates are read at OD₆₀₀ on a Molecular Devices SpectraMax Plus Spectrophotometer. Average OD₆₀₀ values are computed using SOFTmax Pro Software (Molecular Devices) and MIC values are determined by linear regression analysis using Prism version 3.02 (Graph Pad Software, San Diego). The MIC is defined as the concentration of compound required to produce an OD₆₀₀ reading equivalent to 10% of the vehicle control reading.

### Results of the Biological Testing

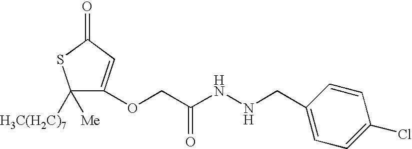

9

| | | | |
|---|---|---|---|
| FAS (IC₅₀) | ¹⁴C (IC₅₀) | XTT (IC₅₀) | XTT (IC₅₀) |
| Neg | 12.7 ± 3.7 ug/ml | 6.0 ± 0.8 ug/ml | 14.9 ug/ml (3T3) |
| 237 ± 43 ug/ml | | 8.8 ug/ml (OV) | 7.3 ug/ml |
| | | | (SKBR) |
| | Cr. Violet (IC₅₀) | 4.7 ug/ml (H) | 9.4 ug/ml (231) |
| | <5 ug/ml | 4.5 ug/ml (RKO) | 4.3 ug/ml (MRC) |

| | Weight Loss |
|---|---|
| | 60 mg/kg: 1.4% (day 2) |
| FAO SC 150 | FAO MAX |
| Neg | 107% |
| | @ 0.195 ug/ml |

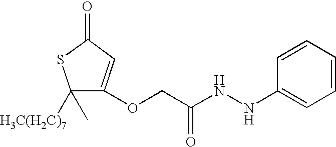

10

| | | | |
|---|---|---|---|
| FAS (IC₅₀) | ¹⁴C (IC₅₀) | XTT (IC₅₀) | Cr. Violet (IC₅₀) |
| Not Tested | 7.2 ug/ml | 6.9 μg/ml (M) | 8.4 ug/ml (H) |
| | | 13.2 (OV) | |

| | Weight Loss |
|---|---|
| | Not Tested |
| FAO SC 150 | FAO Max |
| Neg | 126% at |
| | 6.25 ug/ml |

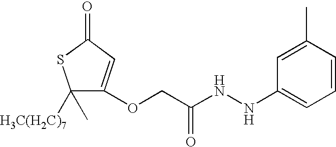

11

| | | | |
|---|---|---|---|
| FAS (IC₅₀) | ¹⁴C (IC₅₀) | XTT (IC₅₀ ) | Cr. Violet (IC₅₀) |
| Not Tested | 12.0 ug/ml | 9.0 μg/ml (M) | 21.9 ug/ml (H) |
| | | 10.1 ml (OV) | |

| | Weight Loss |
|---|---|
| | Not Tested |
| FAO SC 150 | FAO Max |
| Neg | 116% @ |
| | 1.56 ug/ml |

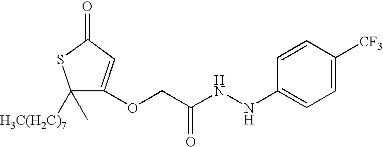

12

| | | | |
|---|---|---|---|
| FAS (IC₅₀) | ¹⁴C (IC₅₀) | XTT (IC₅₀) | Cr. Violet (IC₅₀) |
| Not Tested | 11.3 ug/ml | 5.7 μg/ml (M) | 4.8 μg/ml (H) |
| | | 9.6 ml (OV) | |

| | Weight Loss |
|---|---|
| | 60 mg/kg: 2.7% (day 2) |
| FAO SC 150 | FAO Max |
| Neg | 118% at |
| | 1.56 μg/ml |

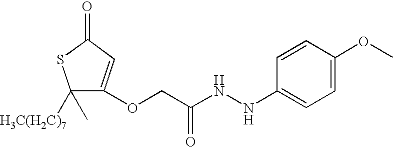

13

| | | | |
|---|---|---|---|
| FAS IC(hd 50) | ¹⁴C (IC₅₀) | XTT (IC₅₀) | Cr. Violet (IC₅₀) |
| Not Tested | 21.3 ug/ml | 9.0 μg/ml (M) | 12.3 μg/ml (H) |
| | | 18.0 μg/ml (OV) | |

| | Weight Loss |
|---|---|
| | Not Tested |
| FAO SC 150 | FAO Max |
| Neg | 103% at |
| | 0.098 μg/ml |

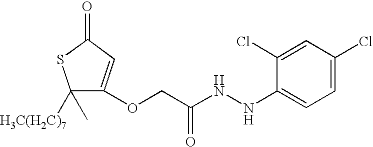

14

| | | | |
|---|---|---|---|
| FAS (IC₅₀) | ¹⁴C (IC₅₀) | XTT (IC₅₀) | Cr. Violet (IC₅₀) |
| Not Tested | 18.3 ug/ml | 6.9 μg/ml (M) | 7.3 μg/ml (H) |
| | | 12.8 μg/ml (OV) | |

| | Weight Loss |
|---|---|
| | Not Tested |
| FAO SC 150 | FAO Max |
| Neg | 100% at |
| | 0.098 μg/ml |

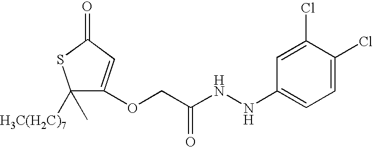

15

| | | | |
|---|---|---|---|
| FAS (IC₅₀) | ¹⁴C (IC₅₀) | XTT (IC₅₀) | Cr. Violet (IC₅₀) |
| Not Tested | 13.3 ug/ml | 11.4 μg/ml (M) | 8.5 μh/ml (H) |
| | | 15.2 μg/ml (OV) | |

| | Weight Loss |
|---|---|
| | Not Tested |
| FAO SC 150 | FAO Max |
| Neg | 100% at |
| | 0.395 μg/ml |

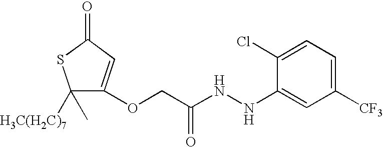

16

| | | | |
|---|---|---|---|
| FAS (IC₅₀) | ¹⁴C (IC₅₀) | XTT (IC₅₀) | Cr. Violet (IC₅₀) |
| Not Tested | 16.5 ug/ml | 6.0 μg/ml (M) | 5.8 μg/ml (H) |
| | | 7.2 μg/ml (OV) | |

| | Weight Loss |
|---|---|
| | 60 mg/kg: 1.7 % (day 3) |
| FAO SC 150 | FAO Max |
| Neg | 137% @ |
| | 6.25 ug/ml |

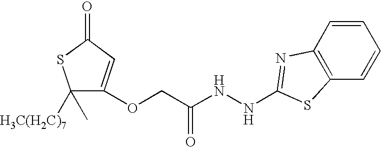

17

| | | | |
|---|---|---|---|
| FAS (IC₅₀) | ¹⁴C (IC₅₀) | XTT (IC₅₀) | Cr. Violet (IC₅₀) |
| Not Tested | Neg (>80 ug/ml) | 74.0 ug/ml (M) | 17.8 ug/ml (H) |
| | | 38.7 ml (OV) | |

| | Weight Loss |
|---|---|
| | Not Tested |
| FAO SC 150 | FAO Max |
| 4.9 ug/ml | 167% @ |
| | 6.25 ug/ml |

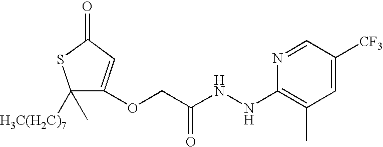

18

| | | | |
|---|---|---|---|
| FAS (IC₅₀) | ¹⁴C (IC₅₀) | XTT (IC₅₀) | Cr. Violet (IC₅₀) |
| Not Tested | 15.4 ug/ml | 5.7 ug/ml (M) | 5.8 ug/ml (H) |
| | | 13.3 μg/ml (OV) | |

| | Weight Loss |
|---|---|
| | 60 mg/kg: 0.6% (day 2) |
| FAO SC 150 | FAO Max |
| 1.6 ug/ml | 149% @ |
| | 1.56 ug/ml |

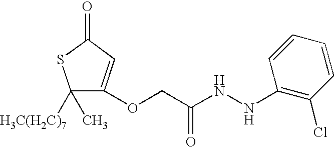

19

| | | | |
|---|---|---|---|
| FAS (IC₅₀) | ¹⁴C (IC₅₀) | XTT (IC₅₀) | Cr. Violet (IC₅₀) |
| Neg | 39.8 ± 12.7 ug/ml | 6.1 ± 0.3 μg/ml | 7.6 μg/ml |

| | 9.4 μg/ml (OV) |
|---|---|
| | Weight Loss |
| | 60 mg/kg: +3.0% (day 1) |
| FAO SC 150 | FAO MAX |
| Neg | 90% @ |
| | 0.125 ug/ml |

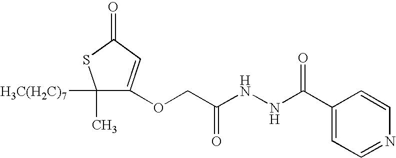

20

| | | | |
|---|---|---|---|
| FAS (IC₅₀) | ¹⁴C (IC₅₀) | XTT (IC₅₀) | Cr. Violet (IC₅₀) |
| Neg | 17.4 ug/ml | 22.5 ± 2.0 μg/ml | 18.2 μg/ml |
| | | 33.0 ± 9.1 μg/ml | |

| | Weight Loss |
|---|---|
| FAO SC 150 | FAO MAX |
| Neg | 89% @ |
| | 0.125 ug/ml |

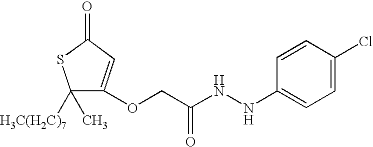

21

| | | | |
|---|---|---|---|
| FAS (IC₅₀) | ¹⁴C (IC₅₀) | XTT (IC₅₀) | Cr. Violet (IC₅₀) |
| Neg | Not Tested | 9.8 μg/ml (M) | 5.3 Not Tested |
| | | 11.2 ml (OV) | |

| | Weight Loss |
|---|---|
| | Not Tested |
| FAO SC 150 | FAO MAX |
| Neg | 104% at |
| | 0.024 μg/ml |

1-25. (canceled)

26. A compound of formula: 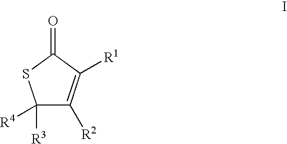
wherein:
R¹ is selected from the group consisting of H, C₁-C₂₀ alkyl, cycloalkyl, alkenyl, aryl, arylalkyl, alkylaryl, —OCH₃, —OC(O)CH₃ and —OC(O)CF₃;
R² is —OCH₂C(O)NHNH—R⁵, wherein R⁵ is selected from the group consisting of
(a) a phenyl group, optionally substituted with one or more of a halogen or a C₁-C₈ alkyl, which is optionally substituted with one or more of a first substitution group selected from the group consisting of a halogen, —OH, and —OR⁶, wherein R⁶ is C₁-C₈ alkyl, optionally substituted with one or more halogens;
(b) a 2-, 3-, or 4-pyridyl, optionally substituted with one or more of a second substitution group selected from the group consisting of a halogen, —OH, and —OR⁶, where R⁶ is C₁-C₈ alkyl, optionally substituted with one or more halogens;
(c) a heterocycle selected from the group consisting of imidazole, thiazole, benzimidizole, benzoxazole, benzthiazole, tetrazole, triazole, and aminothiazole; and
(d) −C(O)R⁷, where R⁷ is selected from the group consisting of a C₁-C₂₀ alkyl, cycloalkyl, alkenyl, aryl, arylalkyl, alkylaryl, and heterocycle, which is selected from the group consisting of pyridyl, imidazole, thiazole, benzimidizole, benzoxazole, benzthiazole, tetrazole, triazole, and aminothiazole; and
R³ and R⁴ are independently selected from the group consisting of C₁-C₂₀ alkyl, cycloalkyl, alkenyl, aryl, arylalkyl, and alkylaryl;
with the proviso that when R¹ is —H, —OCH₃, or —OC(O)CF₃ and R³ is —(CH₂)₇CH₃, then R² is not —OCH₂C(O)NHNH—R⁵, where R⁵ is -p-C₆H₄Cl, —C(O)CH₃, or

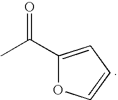

27. A compound according to claim 26, wherein R¹ is H.

28. A compound according to claim 26, wherein R⁵ is selected from the group consisting of C₁-C₁₀ alkyl, cycloalkyl, alkenyl, aryl, arylalkyl, and alkylaryl.

29. A compound according to claim 26, wherein R³ is —H or —CH₃.

30. A compound according to claim 26, wherein R⁴ is n-C₆-C₈ alkyl.

31. A compound according to claim 26, wherein the compound is selected from the group consisting of

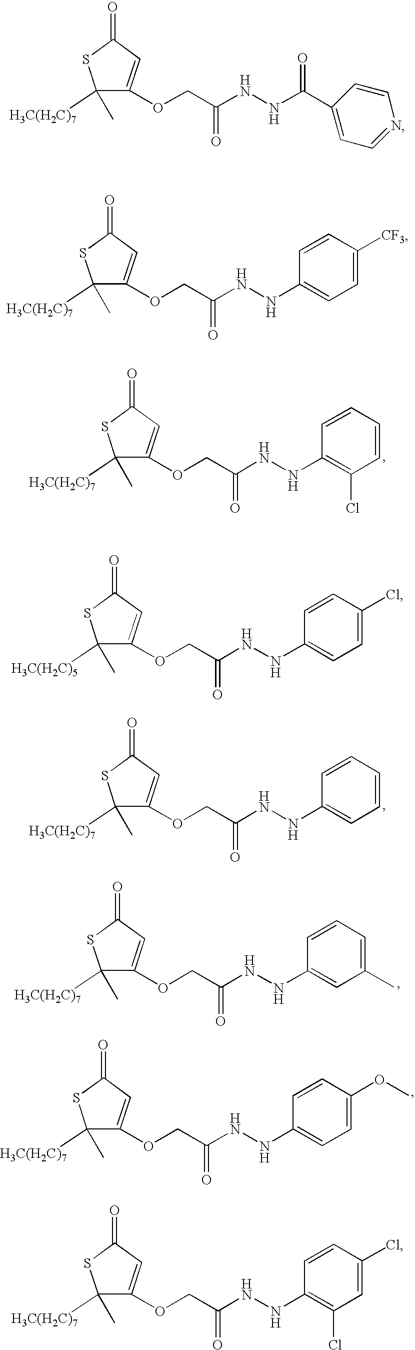

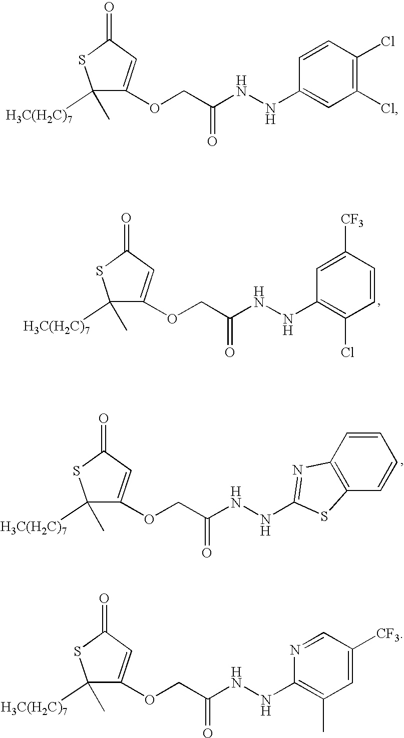

32. A pharmaceutical composition comprising a pharmaceutical diluent and a compound of formula I: 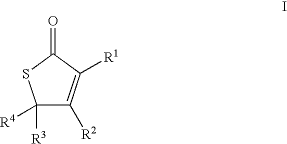
wherein:
R¹ is selected from the group consisting of H, C₁-C₂₀ alkyl, cycloalkyl, alkenyl, aryl, arylalkyl, alkylaryl, —OCH₃, —OC(O)CH₃ and —OC(O)CF₃;
R² is —OCH₂C(O)NHNH—R⁵, wherein R⁵ is selected from the group consisting of
(a) a phenyl group, optionally substituted with one or more of a halogen or a C₁-C₈ alkyl, which is optionally substituted with one or more of a first substitution group selected from the group consisting of a halogen, —OH, and —OR⁶, wherein R⁶ is C₁-C₈ alkyl, optionally substituted with one or more halogens;
(b) a 2-, 3-, or 4-pyridyl, optionally substituted with one or more of a second substitution group selected from the group consisting of a halogen, —OH, and —OR⁶, where R⁶ is C₁-C₈ alkyl, optionally substituted with one or more halogens;
(c) a heterocycle selected from the group consisting of imidazole, thiazole, benzimidizole, benzoxazole, benzthiazole, tetrazole, triazole, and aminothiazole; and
(d) —C(O)R⁷, where R⁷ is selected from the group consisting of a C₁-C₂₀ alkyl, cycloalkyl, alkenyl, aryl, arylalkyl, alkylaryl, and heterocycle, which is selected from the group consisting of pyridyl, imidazole, thiazole, benzimidizole, benzoxazole, benzthiazole, tetrazole, triazole, and aminothiazole; and
R³ and R⁴ are independently selected from the group consisting of C₁-C₂₀ alkyl, cycloalkyl, alkenyl, aryl, arylalkyl, and alkylaryl;
with the proviso that when R¹ is —H, —OCH₃, or —OC(O)CF₃ and R³ is —(CH₂)₇CH₃, then R² is not —OCH₂C(O)NHNH—R⁵, where R⁵ is -p-C₆H₄Cl, —C(O)CH₃, or

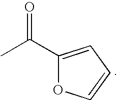

33. A pharmaceutical composition according to claim 32, wherein R¹ is H.

34. A pharmaceutical composition according to claim 32, wherein R⁵ is selected from the group consisting of C₁-C₁₀ alkyl, cycloalkyl, alkenyl, aryl, arylalkyl, and alkylaryl.

35. A pharmaceutical composition according to claim 32, wherein R³ is —H or —CH₃.

36. A pharmaceutical composition according to claim 32, wherein R⁴ is n-C₆-C₈ alkyl.

37. A pharmaceutical composition according to claim 32, wherein the compound is selected from the group consisting of:

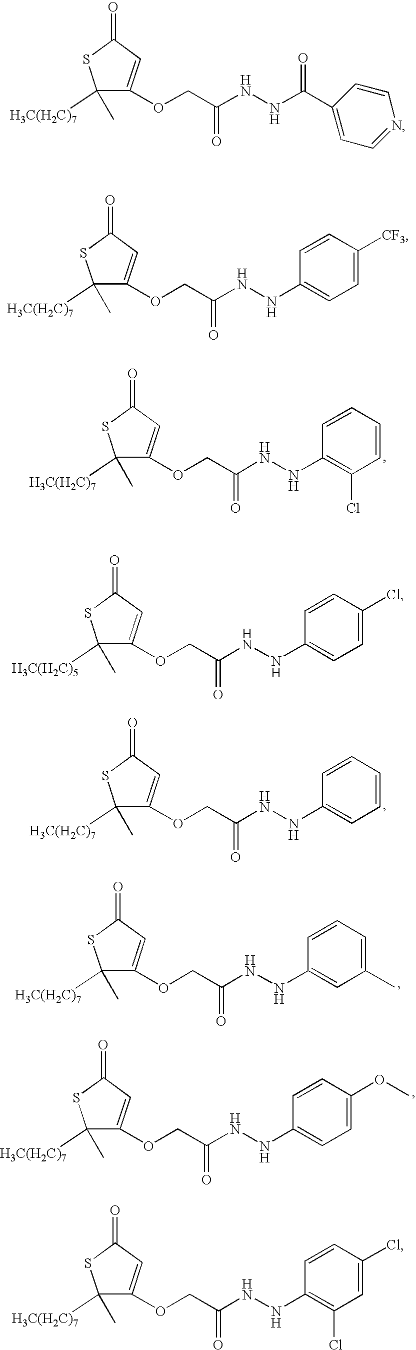

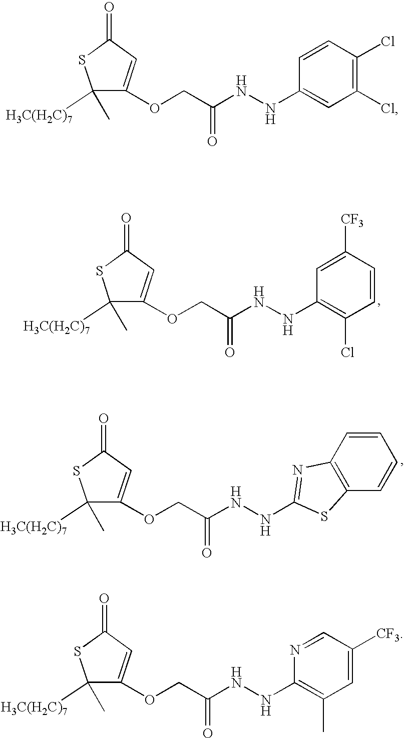

38. A method of treating cancer in a subject, comprising administering an effective amount of a pharmaceutical composition comprising a pharmaceutical diluent and a compound of formula I: 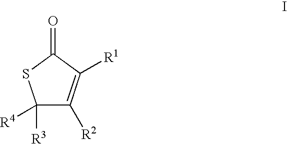
wherein:
R¹ is selected from the group consisting of H, C₁-C₂₀ alkyl, cycloalkyl, alkenyl, aryl, arylalkyl, alkylaryl, —OCH₃, —OC(O)CH₃ and —OC(O)CF₃;
R² is —OCH₂C(O)NHNH—R⁵, wherein R⁵ is selected from the group consisting of
(a) a phenyl group, optionally substituted with one or more of a halogen or a C₁-C₈ alkyl, which is optionally substituted with one or more of a first substitution group selected from the group consisting of a halogen, —OH, and —OR⁶, wherein R⁶ is C₁-C₈ alkyl, optionally substituted with one or more halogens;
(b) a 2-, 3-, or 4-pyridyl, optionally substituted with one or more of a second substitution group selected from the group consisting of a halogen, —OH, and —OR⁶, where R⁶ is C₁-C₈ alkyl, optionally substituted with one or more halogens;
(c) a heterocycle selected from the group consisting of imidazole, thiazole, benzimidizole, benzoxazole, benzthiazole, tetrazole, triazole, and aminothiazole; and
(d) —C(O)R⁷, where R⁷ is selected from the group consisting of a C₁-C₂₀ alkyl, cycloalkyl, alkenyl, aryl, arylalkyl, alkylaryl, and heterocycle, which is selected from the group consisting of pyridyl, imidazole, thiazole, benzimidizole, benzoxazole, benzthiazole, tetrazole, triazole, and aminothiazole; and
R³ and R⁴ and independently selected from the group consisting of C₁-C₂₀ alkyl, cycloalkyl, alkenyl, aryl, arylalkyl, and alkylaryl;
with the proviso that when R¹ is —H, —OCH₃, or —OC(O)CF₃ and R³ is —(CH₂)₇CH₃, then R² is not —OCH₂C(O)NHNH—R⁵, where R⁵ is -p-C₆H₄Cl, —C(O)CH₃, or

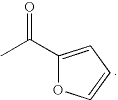

39. The method of claim 38, wherein the subject is a human.

40. The method of claim 39 wherein the pharmaceutical composition comprises a compound selected from the group consisting of:

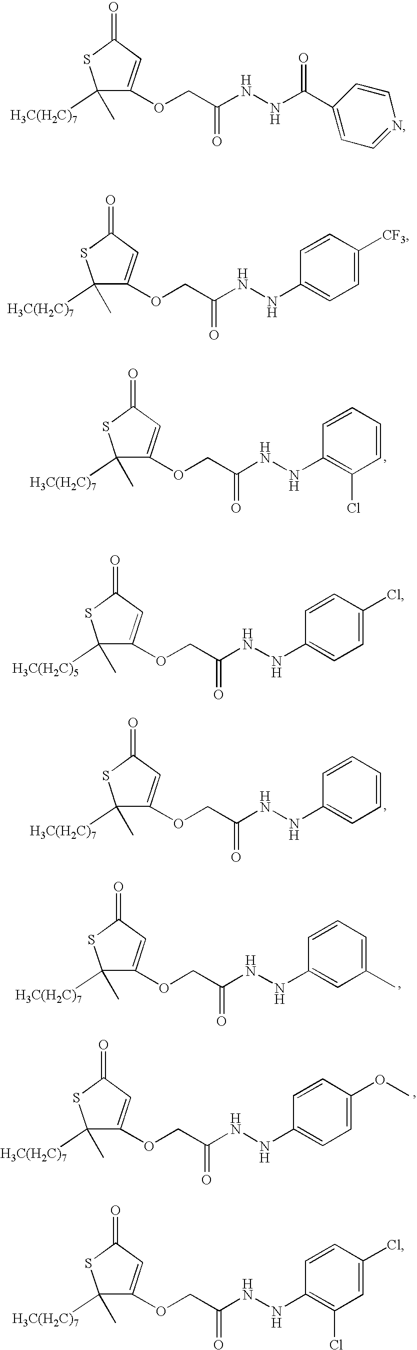

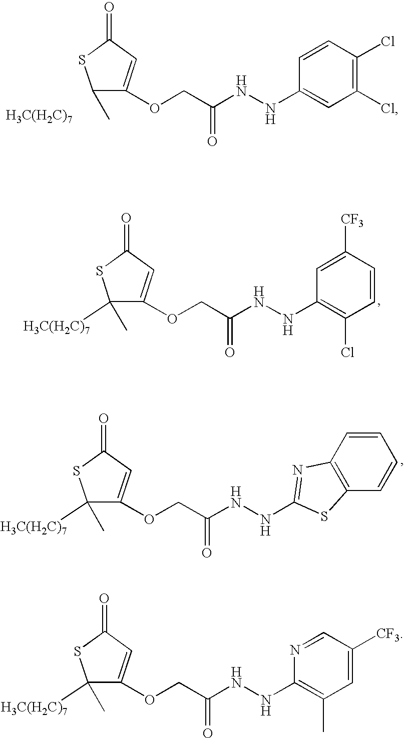

41. The method of claim 38, wherein the subject is an animal.

42. The method of claim 41, wherein the pharmaceutical composition comprises a compound selected from the group consisting of:

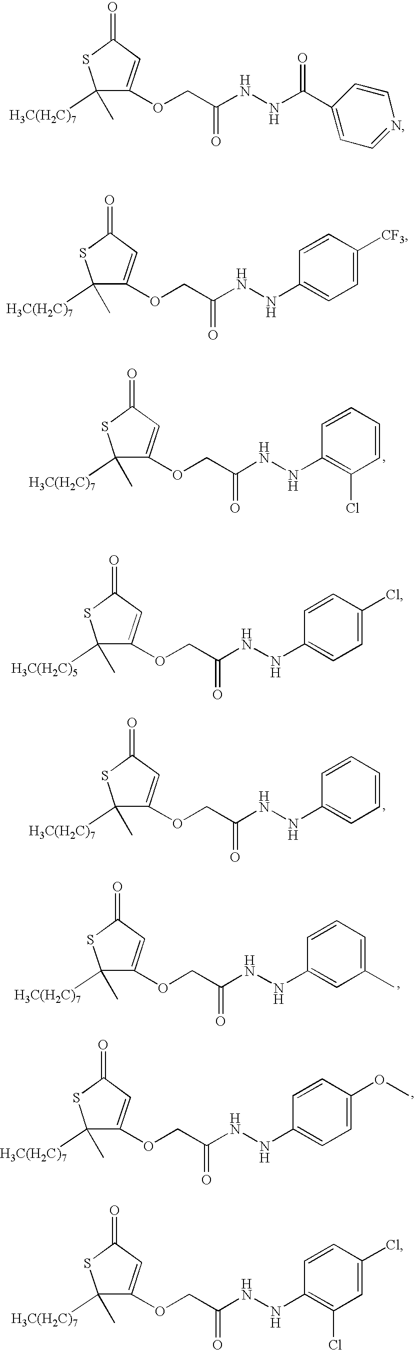

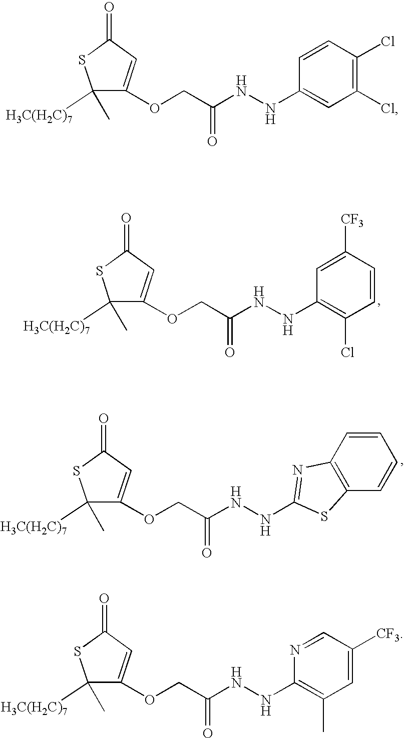

43. A method of inhibiting fatty acid synthase activity in a subject comprising administering an effective amount of a pharmaceutical composition comprising a pharmaceutical diluent and a compound of formula I: 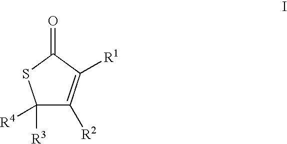
wherein:
R¹ is selected from the group consisting of H, C₁-C₂₀ alkyl, cycloalkyl, alkenyl, aryl, arylalkyl, alkylaryl, —OCH₃, —OC(O)CH₃ and —OC(O)CF₃;
R² is —OCH₂C(O)NHNH—R⁵, wherein R⁵ is selected from the group consisting of
(a) a phenyl group, optionally substituted with one or more of a halogen or a C₁-C₈ alkyl, which is optionally substituted with one or more of a first substitution group selected from the group consisting of a halogen, —OH, and —OR⁶, wherein R⁶ is C₁-C₈ alkyl, optionally substituted with one or more halogens;
(b) a 2-, 3-, or 4-pyridyl, optionally substituted with one or more of a second substitution group selected from the group consisting of a halogen, —OH, and —OR⁶, where R⁶ is C₁-C₈ alkyl, optionally substituted with one or more halogens;
(c) a heterocycle selected from the group consisting of imidazole, thiazole, benzimidizole, benzoxazole, benzthiazole, tetrazole, triazole, and aminothiazole; and
(d) —C(O)R⁷, where R⁷ is selected from the group consisting of a C₁-C₂₀ alkyl, cycloalkyl, alkenyl, aryl, arylalkyl, alkylaryl, and heterocycle, which is selected from the group consisting of pyridyl, imidazole, thiazole, benzimidizole, benzoxazole, benzthiazole, tetrazole, triazole, and aminothiazole; and
R³ and R⁴ and independently selected from the group consisting of C₁-C₂₀ alkyl, cycloalkyl, alkenyl, aryl, arylalkyl, and alkylaryl;
with the proviso that when R¹ is —H, —OCH₃, or —OC(O)CF₃ and R³ is —(CH₂)₇CH₃, then R² is not —OCH₂C(O)NHNH—R⁵, where R⁵ is -p-C₆H₄Cl, —C(O)CH₃, or

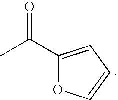

44. The method of claim 43, wherein the subject is a human.

45. The method of claim 43, wherein the subject is an animal.

46. A method of inhibiting growth of invasive microbial cells in a subject comprising the administration of an effective amount of a pharmaceutical composition comprising a pharmaceutical diluent and a compound of formula I: 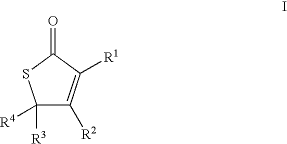
wherein:
R¹ is selected from the group consisting of H, C₁-C₂₀ alkyl, cycloalkyl, alkenyl, aryl, arylalkyl, alkylaryl, —OCH₃, —OC(O)CH₃ and —OC(O)CF₃;
R² is —OCH₂C(O)NHNH—R⁵, wherein R⁵ is selected from the group consisting of
(a) a phenyl group, optionally substituted with one or more of a halogen or a C₁-C₈ alkyl, which is optionally substituted with one or more of a first substitution group selected from the group consisting of a halogen, —OH, and —OR⁶, wherein R⁶ is C₁-C₈ alkyl, optionally substituted with one or more halogens;
(b) a 2-, 3-, or 4-pyridyl, optionally substituted with one or more of a second substitution group selected from the group consisting of a halogen, —OH, and —OR⁶, where R⁶ is C₁-C₈ alkyl, optionally substituted with one or more halogens;
(c) a heterocycle selected from the group consisting of imidazole, thiazole, benzimidizole, benzoxazole, benzthiazole, tetrazole, triazole, and aminothiazole; and
(d) —C(O)R⁷, where R⁷ is selected from the group consisting of a C₁-C₂₀ alkyl, cycloalkyl, alkenyl, aryl, arylalkyl, alkylaryl, and heterocycle, which is selected from the group consisting of pyridyl, imidazole, thiazole, benzimidizole, benzoxazole, benzthiazole, tetrazole, triazole, and aminothiazole; and
R³ and R⁴ and independently selected from the group consisting of C₁-C₂₀ alkyl, cycloalkyl, alkenyl, aryl, arylalkyl, and alkylaryl;
with the proviso that when R¹ is —H, —OCH₃, or —OC(O)CF₃ and R³ is —(CH₂)₇CH₃, then R² is not —OCH₂C(O)NHNH—R⁵, where R⁵ is -p-C₆H₄Cl, —C(O)CH₃, or

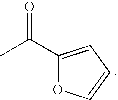

47. The method of claim 46, wherein the subject is a human.

48. The method of claim 47, wherein the compound is selected from the group consisting of:

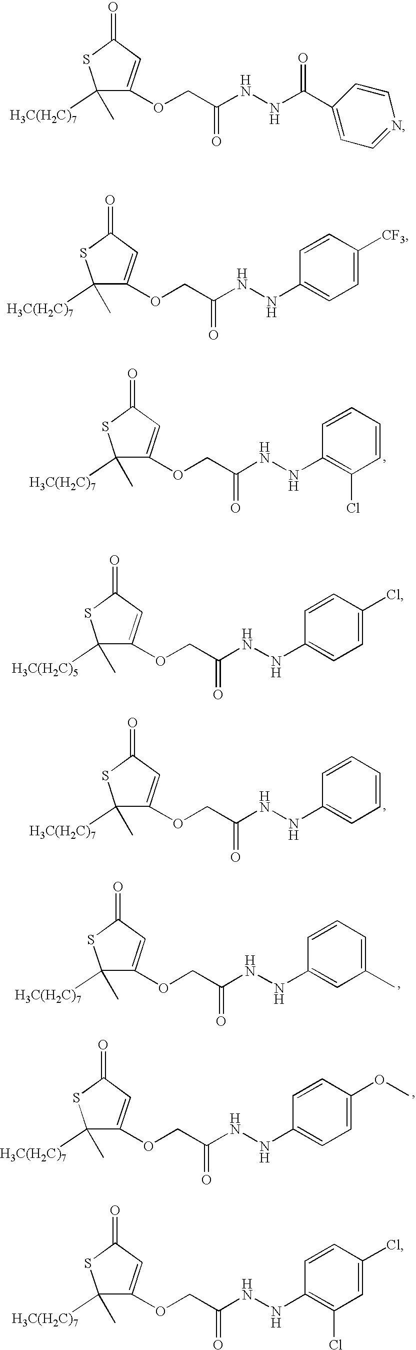

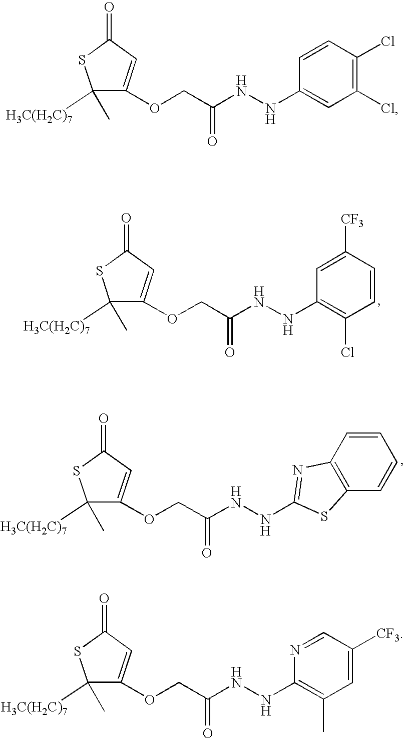

49. The method of claim 46, wherein the subject is an animal.

50. The method of claim 49, wherein the compound is selected from the group consisting of: 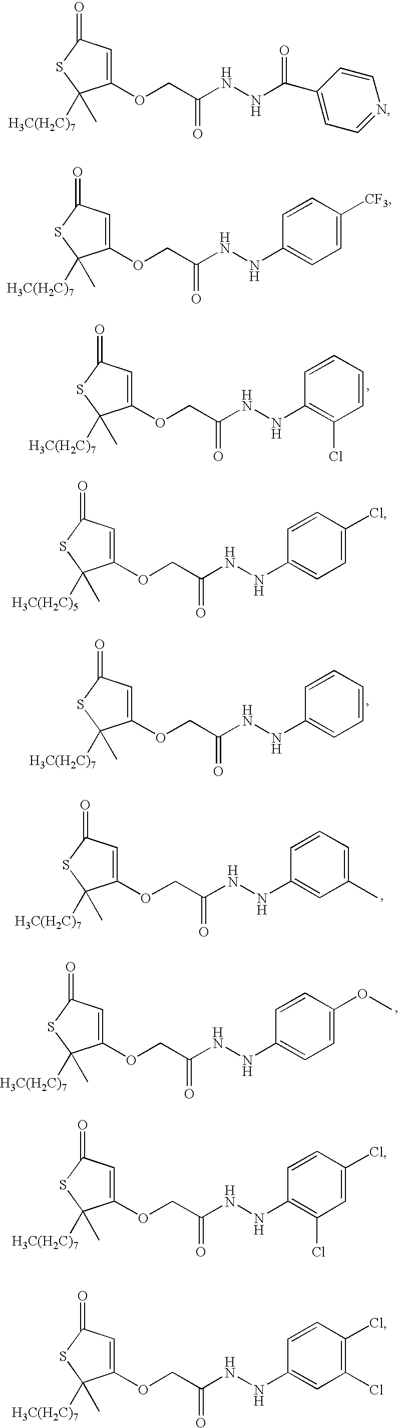 [chemical structure]
